(19) United States
(12) Patent Application Publication
(10) Pub. No.: US 2005/0015206 A1
(43) Pub. Date: Jan. 20, 2005

(54) Nucleic acid detection assay control genes

(76) Inventor: Uwe Scherf, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/483,889

(22) PCT Filed: Jul. 12, 2002
(86) PCT No.: PCT/US02/21821

Related U.S. Application Data
(60) Provisional application No. 60/305,154, filed on Jul. 16, 2001.

(51) Int. Cl. G01N 33/48

(52) U.S. Cl. 702/19

(57) ABSTRACT

The present invention includes methods of normalizing quantitative and non-quantitative nucleic acid detection as-says by monitoring control genes. These methods have applicability across a broad spectrum of hybridization format.

### RELATED APPLICATIONS

This application claims priority to U.S. Provisional Application 60/305,154 (filed Jul. 16, 2001), which is herein incorporated by reference in its entirety.

### FIELD OF THE INVENTION

The invention relates generally to control genes that may be utilized for normalizing hybridization and/or amplification reactions.

### BACKGROUND OF THE INVENTION

Nucleic acid hybridization and other quantitative nucleic acid detection assays are routinely used in medical and biotechnological research and development, diagnostic testing, drug development and forensics. Such technologies have been used to identify genes which are up- or down-regulated in various disease or physiological states, to analyze the roles of the members of cellular signaling cascades and to identify druggable targets for various disease and pathology states.

Examples of technologies commonly used for the detection and/or quantification of nucleic acids include northern blotting (Krumlauf (1994) Mol Biotechnol 2(3), 227-242), ill situ hybridization (Parker & Barnes (1999) Methods Mol Biol. 106, 247-83), RNAse protection assays (Hod (1992) Biotechniques 13(6), 852-854; Saccomanno et al. (1992) Biotechniques 13(6):846-50), microarrays, and reverse transcription polymerase chain reaction (RT-PCR) (see Bustin, (2000) Journal of Molecular Endocrinology 25, 169-193).

The reliability of these nucleic acid detection methods depend on the availability of accurate means for accounting for variations between analyses. For example, variations in hybridization conditions, label intensity, reading and detector efficiency, sample concentration and quality, background effects, and image processing effects each contribute to signal heterogeneity. Hegde et al. (2000) Biotechniques 29(3): 548-562; Berger et al. (2000) WO 00/04188. Normalization procedures used to overcome these variations often rely on control hybridizations to housekeeping genes such as β-actin, glyceraldehyde-3-phosphate dehydrogenase, and the transferrin receptor gene. Eickhoffet al. (1999) Nucleic Acids Research 27(22): e33; Spiess et al. (1999) Biotechniques 26(1): 46-50. These methods, however, generally do not provide the signal linearity sufficient to detect small but significant changes in transcription or gene expression. Spiess et al. (1999) Biotechniques 26(1): 46-50. In addition, the steady state levels of many housekeeping genes are susceptible to alterations in expression levels that are dependent on cell differentiation, nutritional state, specific experimental and stimulation protocols. Eickhoffet al. (1999) Nucleic Acids Research 27(22): e33; Spiess et al. (1999) Biotechniques 26(1): 46-50; Hegde et al. (2000)Biotechniques 29(3): 548-562; and Berger et al. (2000) WO 00/04188. Consequently, there exists a need for the identification and use of additional genes that may serve as effective controls in nucleic acid detection assays.

### SUMMARY OF THE INVENTION

The present invention includes methods of identifying at least one gene that is consistently expressed across different cell or tissue types in an organism, comprising: preparing gene expression profiles for different cell or tissue types from the organism; calculating a coefficient of variation for at least one gene in each of the profiles across the different cell or tissue types; and selecting any gene whose coefficient of variation indicates that the gene is consistently expressed across the different cell or tissue types. The coefficient of variation may be less than about 40% and the methods may comprise creating gene expression profiles for about 10, 25, 50, 100 or more different cell or tissue types. The gene expression profiles may be prepared be querying a gene expression database.

The invention also includes a set of probes comprising at least two probes that specifically hybridize to a control gene identified by the methods of the invention. Such sets of probes may comprise probes that specifically hybridize to at least about 10, 25, 50 or 100 control genes. In some formats, the sets of probes are attached to a solid substrate such as a microarray or chip.

The invention also includes methods of normalizing the data from a nucleic acid detection assay comprising: detecting the expression level for at least one gene in a nucleic acid sample; and normalizing the expression of said at least one gene with the detected expression of at least one control gene identified by the method of the invention. The number of control genes used to normalize gene expression data may comprise about 10, 25, 50, 100 or more of the control genes herein identified.

In another embodiment, the invention includes a set of probes comprising at least two probes that specifically hybridize to a gene of Table 1 or Table 2. The set may comprise at least about 10, 25, 50, 100 or more the control genes of Table 1 or Table 2. The sets of probes may or may not be attached to a solid substrate such as a chip.

The invention, in another embodiment, includes methods of normalizing the data from a nucleic acid detection assay comprising: detecting the expression level for at least one gene in a nucleic acid sample; and normalizing the expression of said at least one gene with the detected expression of at least one control gene of Table 1 or Table 2. The number of control genes used to normalize gene expression data may comprise about 10, 25, 50, 100 or more of the control genes herein identified.

### DETAILED DESCRIPTION

The present Inventors have identified control genes that may be monitored in nucleic acid detection assays and whose expression levels may be used to normalize gene expression data. Normalization of gene expression data from a cell or tissue sample with the expression level(s) of the identified genes allows the accurate assessment of the expression level(s) for genes that are differentially regulated between samples, tissues, treatment conditions, etc. These genes may be used across a broad spectrum of assay formats, but are particularly useful in microarray or hybridization based assay formats.

A. Nucleic Acid Detection Assay Controls

1. Selection of Control Genes

As used herein, the genes and nucleic acids of Tables 1 and 2 are referred to as “control genes.”

Control genes of the invention are produced by a method comprising preparing gene expression profiles (a representation of the expression level for at least one gene, preferably 10, 50, 100 or more, or most preferably nearly all or all expressed genes in a sample) from a variety of cell or tissue types, measuring the level of expression for at least one gene in each of the gene expression profiles to produce gene expression data, calculating a coefficient of variation from the gene expression data for each gene and selecting genes whose coefficient of variation indicates that the gene is consistently expressed at about the same level in the different cell or tissue types.

The gene expression profile may be produced by any means of quantifying gene expression for at least one gene in the tissue or cell sample. In preferred methods, gene expression is quantified by a method selected from the group consisting of a hybridization assay or an amplification assay. Hybridization assays may be any assay format, such as this described below, that relies on the hybridization of a probe or primer to a nucleic acid molecule in the sample. Such formats include, but are not limited to, differential display formats and microarray hybridization, including microarrays produced in chip format. Amplification assays include, but are not limited to, quantitative PCR, semiquantitative PCR and assays that rely on amplification of nucleic acids subsequent to the hybridization of the nucleic acid to a probe or primer. Such assays include the amplification of nucleic acid molecules from a sample that are bound to a microarray or chip.

In other circumstances, gene expression profiles may be produced by querying a gene expression database comprising expression results for genes from various cell or tissue samples. The gene expression results in the database may be produced by any available method, such as differential display methods and microarray-based hybridization methods. The gene expression profile is typically produced by the step of querying the database with the identity of a specific cell or tissue type for the genes that are expressed in the cell or tissue type and/or the genes that are differentially regulated compared to a control cell or tissue sample. Available databases include, but are not limited to, the Gene Logic GeneExpress® database, the Gene Expression Omnibus gene expression and hybridization array repository available through NCBI (www.ncbi.nln.nih.gov/entrez) and the SAGE™ gene expression database.

The cell or tissue samples that are used to prepare gene expression profiles may include any cell or tissue sample available. Such samples include, but are not limited to, tissues removed as surgical samples, diseased or normal tissues, in vitro or in vivo grown cells, cell culture and cells or tissues exposed to an agent such as a toxin. The number of samples required to calculate a coefficient of variation is variable, but may include about 10, 25, 50, 100, 200, 500 or more cell or tissue samples. The cell or tissue samples may be derived from an animal or plant, preferably a mammal. In some instances, the cell or tissue samples may be human, canine (dog), mouse or rat in origin.

The coefficient of variation may be calculated from raw expression data or from data that has been normalized to control for the mechanics of hybridization, such as data normalized or controlled for background noise due to non-specific hybridization. Such data typically includes, but is not limited to, fluorescence readings from microarray based hybridizations, densitometry readings produced from assays that rely on radiological labels to detect and quantify gene expression and data produced from quantitative or semi-quantitative amplification assays.

The coefficient of variation (% CV) is typically calculated by calculating a mean value for the expression level of a given gene across a number of samples and calculating the standard deviation (SD) from that mean. The % CV may be calculated by the following equation: % CV=SD/Mean×100. Genes with a % CV of less than about 50% and preferably less than about 40%, may be selected as control genes or are considered as genes that are consistently expressed across the different cell or tissue types tested.

As used herein, “background” refers to signals associated with non-specific binding (cross-hybridization). In addition to cross-hybridization, background may also be produced by intrinsic fluorescence of the hybridization format components themselves.

“Bind(s) substantially” refers to complementary hybridization between an oligonucleotide probe and a nucleic acid sample and embraces minor mismatches that can be accommodated by reducing the stringency of the hybridization media to achieve the desired detection of the nucleic acid sample.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

2. Preparation of Controls Genes, Probes and Primers

The control genes listed in Tables 1 and 2 may be obtained from a variety of natural sources such as organisms, organs, tissues and cells. The sequences of known genes are in the public databases. The GenBank Accession Number corresponding to the Normalization Control Genes can be found in the third column of the Tables under “Exemplar Seq: Accession.” The sequences of the genes in GenBank (http://www.ncbi.nlin.nih.gov/) are herein incorporated by reference in their entirety.

Probes or primers for the nucleic acid detection assays described herein that specifically hybridize to a control gene may be produced by any available means. For instance, probe sequences may be prepared by cleaving DNA molecules produced by standard procedures with commercially available restriction endonucleases or other cleaving agent. Following isolation and purification, these resultant normalization control gene fragments can be used directly, amplified by PCR methods or amplified by replication or expression from a vector.

Control genes and control gene probes or primers (i.e., synthetic oligo- and polynucleotides) are most easily synthesized by chemical techniques, for example, the phosphotriester method of Matteucci, et al. ((1981) J. Am. Chem. Soc. 103: 3185-3191) or using automated synthesis methods using the GenBank sequences disclosed in Tables 1 and 2. In addition, larger nucleic acids can readily be prepared by well known methods, such as synthesis of a group of oligonucleotides that define various modular segments of the normalization control genes and normalization control gene segments, followed by ligation of oligonucleotides to build the complete nucleic acid molecule.

B. Normalization Methods

Gene expression data produced from the control genes in a given sample or samples may be used to normalize the gene expression data from other genes using any available arithmatic or calculative means. Such methods include, but are not limited, methods of data analysis described by Hegde et al. (2000)Biotechiniques 29(3): 548-562; Winzeller et al. (1999) Meth. Enzymol. 306(1): 3-18; Tkatchenko et al. (2000) Biochimica et Biophysica Acta 1500: 17-30; Berger et al. (2000) WO 00/04188; Schuchhardt et al. (2000) Nucleic Acids Research 28(10): e47; Eickhoffet al. (1999) Nucleic Acids Research 27(22): e33. Micro-array data analysis and image processing software packages and protocols, including normalization methods, are also available from BioDiscovery (http://www.biodiscovery.com/), Silicon Graphics (http://www.sigenetics.com), Spotfire (http://www.spotfire.com/), Stanford University (http://rana.Stanford.EDU/software/), National Human Genome Research Institute (http://www.nhgri.nih.gov/DIR/LCG/15K/HTML/img_analysis.html), TIGR (http://www.tigr.org/softlab/), and Affymetrix (affy and maffy paclkages), among others.

C. Assay or Hybridization Formats

The control genes of the present invention may be used in any nucleic acid detection assay format, including solution-based and solid support-based assay formats. As used herein, “hybridization assay format(s)” refer to the organization of the oligonucleotide probes relative to the nucleic acid sample. The hybridization assay formats that may be used with the control genes and methods of the present invention include assays where the nucleic acid sample is labeled with one or more detectable labels, assays where the probes are labeled with one or more detectable labels, and assays where the sample or the probes are immobilized. Hybridization assay formats include but are not limited to: Northern blots, Southern blots, dot blots, solution-based assays, branched-DNA assays, PCR, RT-PCR, quantitative or semi-quantitative RT-PCR, microarrays and biochips.

As used herein, “nucleic acid hybridization” simply involves contacting a probe and nucleic acid sample under conditions where the probe and its complementary target can form stable hybrid duplexes through complementary base pairing (see Lockhart et al., (1999) WO 99/32660). The nucleic acids that do not form hybrid duplexes are then washed away leaving the hybridized nucleic acids to be detected, typically through detection of an attached detectable label.

It is generally recognized that nucleic acids are denatured by increasing the temperature or decreasing the salt concentration of the buffer containing the nucleic acids.

Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPET at 37° C. to 50° C. until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

As used herein, the term “stringent conditions” refers to conditions under which a probe will hybridize to a complementary control nucleic acid, but with only insubstantial hybridization to other sequences. Stringent conditions are sequence-dependent and will be different under different circumstances. Longer sequences hybridize specifically at higher temperatures. Generally, stringent conditions are selected to be about 5° C. lower than the thermal melting point (Tₘ) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotide). Stringent conditions may also be achieved with the addition of destabilizing agents such as formamide.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above that the hybridization pattern is not appreciably altered and which provides adequate signal for the particular oligonucleotide probes of interest.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical residue (e.g., nucleic acid base or amino-acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights. Sequences corresponding to the control genes of Tables 1 and 2 may comprise at least about 70% sequence identity to the GenBank IDS of the genes in the Tables, preferably about 75%, 80% or 85% or more preferably, about 90% or 95% or more identity.

Homology or identity is determined by BLAST (Basic Local Alignment Search Tool) analysis using the algorithm employed by the programs blastp, blastn, blastx, tblastn and tblastx (Karlin et al., (1990) Proc. Natl. Acad. Sci. USA 87, 2264-2268 and Altschul, (1993) J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (1994) Nature Genet. 6, 119-129) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henilcoff et al., (1992) Proc. Natl. Acad. Sci. USA 89, 10915-10919, fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; winkl; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

As used herein a “probe” or “oligonucleotide probe” is defined as a nucleic acid, capable of binding to a nucleic acid sample or complementary control gene nucleic acid through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

Probe arrays may contain at least two or more oligonucleotides that are complementary to or hybridize to one or more of the control genes described herein. Such arrays may also contain oligonucleotides that are complementary or hybridize to at least about 2, 3, 5, 7, 10, 50, 100 or more the genes described herein. Any solid surface to which oligonucleotides or nucleic acid sample can be bound, either directly or indirectly, either covalently or non-covalently, can be used. For example, solid supports for various hybridization assay formats can be filters, polyvinyl chloride dishes, silicon or glass based chips, etc. Glass-based solid supports, for example, are widely available, as well as associated hybridization protocols. (See, e.g., Beattie, WO 95/11755).

A preferred solid support is a high density array or DNA chip. This contains an oligonucleotide probe of a particular nucleotide sequence at a particular location on the array. Each particular location may contain more than one molecule of the probe, but each molecule within the particular location has an identical sequence. Such particular locations are termed features. There may be, for example, 2, 10, 100, 1000, 10,000, 100,000, 400,000, 1,000,000 or more such features on a single solid support. The solid support, or more specifically, the area wherein the probes are attached, may be on the order of a square centimeter.

1. Dot Blots

The control genes listed in Tables 1 and 2 and methods of the present invention may be utilized in numerous hybridization formats such as dot blots, dipstick, branched DNA sandwich and ELISA assays. Dot blot hybridization assays provide a convenient and efficient method of rapidly analyzing nucleic acid samples in a sensitive manner. Dot blots are generally as sensitive as enzyme-linked immunoassays. Dot blot hybridization analyses are well known in the art and detailed methods of conducting and optimizing these assays are detailed in U.S. Pat. No. 6,130,042 and 6,129,828, and Tkatchenlco et al. (2000) Biochimica et Biophysica Acta 1500: 17-30. Specifically, labeled or unlabeled nucleic acid sample is denatured and bound to a membrane (i.e. nitrocellulose), and is then contacted with unlabeled or labeled oligonucleotide probes. Buffer and temperature conditions can be adjusted to vary the degree of identity between the oligonucleotide probes and nucleic acid sample necessary for hybridization.

Several modifications of the basic Dot blot hybridization format have been devised. For example, Reverse Dot blot analyses employ the same strategy as the Dot blot method, except that the oligonucleotide probes are bound to the membrane and the nucleic acid sample is applied and hybridized to the bound probes. Similarly, the Dot blot hybridization format can be modified to include formats where either the nucleic acid sample or the oligonucleotide probe is applied to microtiter plates, microbeads or other solid substrates.

2. Membrane-Based Formats

Although each membrane-based format is essentially a variation of the Dot blot hybridization format, several types of these formats are preferred. Specifically, the methods of the present invention may be used in Northern and Southern blot hybridization assays. Although the methods of the present invention are generally used in quantitative nucleic acid hybridization assays, these methods may be used in qualitative or semi-quantitative assays such as Southern blots, in order to facilitate comparison of blots. Southern blot hybridization, for example, involves cleavage of either genomic or cDNA with restriction endonucleases followed by separation of the resultant fragments on a polyacrylamide or agarose gel and transfer of the nucleic acid fragments to a membrane filter. Labeled oligonucleotide probes are then hybridized to the membrane-bound nucleic acid fragments. In addition, intact cDNA molecules may also be used, separated by electrophoresis, transferred to a membrane and analyzed by hybridization to labeled probes. Northern analyses, similarly, are conducted on nucleic acids, either intact or fragmented, that are bound to a membrane. The nucleic acids in Northern analyses, however, are generally RNA.

3. Arrays.

Any microarray platform or technology may be used to produce gene expression data that may be normalized with the control genes and methods of the invention. Oligonucleotide probe arrays can be made and used according to any techniques known in the art (see for example, Lockhart et al., (1996) Nat. Biotechnol. 14, 1675-1680; McGall et al., (1996) Proc. Nat. Acad. Sci. USA 93, 13555-13460). Such probe arrays may contain at least one or more oligonucleotides that are complementary to or hybridize to one or more of the nucleic acids of the nucleic acid sample and/or the control genes of Tables 1 and 2. Such arrays may also contain oligonucleotides that are complementary or hybridize to at least 2, 3, 5, 7, 10, 50, 100 or more of the control genes listed in Tables 1 and 2.

Control oligonucleotide probes of the invention are preferably of sufficient length to specifically hybridize only to appropriate, complementary genes or transcripts. Typically the oligonucleotide probes will be at least about 10, 12, 14, 16, 18, 20 or 25 nucleotides in length. In some cases longer probes of at least 30, 40, or 50 nucleotides will be desirable. The oligonucleotide probes of high density array chips include oligonucleotides that range from about 5 to about 45 or 5 to about 500 nucleotides, more preferably from about 10 to about 40 nucleotides and most preferably from about 15 to about 40 nucleotides in length. In other particularly preferred embodiments the probes are 20 or 25 nucleotides in length. In another preferred embodiment, probes are double or single strand DNA sequences. The oligonucleotide probes are capable of specifically hybridizing to the control gene nucleic acids in a sample.

One of skill in the art will appreciate that an enormous number of array designs comprising control probes of the invention are suitable for the practice of this invention. The high density array will typically include a number of probes that specifically hybridize to each control gene nucleic acid, e.g. mRNA or cRNA. (See WO 99/32660 for methods of producing probes for a given gene or genes.) Assays and methods comprising control probes of the invention may utilize available formats to simultaneously screen at least about 100, preferably about 1000, more preferably about 10,000 and most preferably about 500,000 or 1,000,000 different nucleic acid hybridizations.

The methods and control genes of this invention may also be used to normalize gene expression data produced using commercially available oligonucleotide arrays that contain or are modified to contain control gene probes or the invention. A preferred oligonucleotide array may be selected from the Affymetrix, Inc. GeneChip® series of arrays which include the GeneChip® Human Genome U95 Set, GeneChip® Hu35K Set, GeneChip®, HuGeneFL Array, GeneChip® Human Cancer G110 Array, GeneChip® Rat Genome U34 Set, GeneChip® Mu19K Set, GeneChip® Mu11K Set, GeneChip® Yeast Genome S98 Array, GeneChip® E. coli Genome Array, GeneChip® Arabidopsis Genome Array, GeneChip® HuSNP™ Probe Array, GeneChip® GenFlex™ Tag Array, GeneChip® HIV PRT Plus Probe Array, GeneChip® P53 Probe Array, GeneChip®, and the CYP450 Probe Array. In another embodiment, an oligonucleotide array may be selected from the Incyte Pharmaceuticals, Inc. GEM™ series of arrays which includes the UniGEM™ V 2.0, Human Genome GEM 1, Human Genome GEM 2, Human Genome GEM 3, Human Genome GEM 4, Human Genome GEM 5, LifeGEM™ 1 Cancer/Signal Peptide, LifeGEM 2 Inflammation/Blood, Mouse GEM 1 Rat GEM 1 Liver/Kidney,Rat GEM 2 Central Nervous System, Rat GEM 3 Liver/Kidney, S. aureus GEM 1, C. albicans GEM 1, and Arabidopsis GEM.

4. RT-PCR

The control genes and methods of the invention may be used in any type of polymerase chain reaction. A preferred PCR format is reverse transciptase polymerase chain reaction (RT-PCR), an in vitro method for enzymatically amplifying defined sequences of RNA (Rappolee et al., (1988) Science 241, 708-712) permitting the analysis of different samples from as little as one cell in the same experiment (See Arubion: RT-PCR: The Basics; M. J. McPherson and S. G. Møller, PCR BIOS Scientific Publishers Ltd Oxford, OX4 1RE (2000); Dieffenbach et al., PCR Primer: A Laboratory Manual Cold Spring Harbor Laboratory Press 1995 for review). One of ordinary skill in the art may appreciate the enormous number of variations in RT-PCR platforms that are suitable for the practice of the invention, including complex variations aimed at increasing sensitivity such as semi-nested (Wasserman et al., (1999) Molecular Diagnostics 4, 21-28), nested (Israeli et al., (1994) Cancer Research 54, 6303-6310; Soeth et al., (1996) International Journal of Cancer 69, 278-282), and even three-step nested (Funaki et al., (1997) Life Sciences 60, 643-652; Funaki et al., (1998) British Journal of Cancer 77, 1327-1332).

In one embodiment of the invention, separate enzymes are used for reverse transcription and PCR amplification. Two commonly used reverse transcriptases, for example, are avian myeloblastosis virus and Moloney murine leukaemia virus. For amplification, a number of thermostable DNA-dependent DNA polymerases are currently available, although they differ in processivity, fidelity, thermal stability and ability to read modified triphosphates such as deoxyuridine and deoxyinosine in the template strand (Adams et al., (1994) Bioorganic and Medicinal Chemistry 2, 659-667; Perler et al., (1996) Advances in Protein Chemistry 48, 377-435). The most commonly used enzyme, Taq DNA polymerase, has a 5′-3′ nuclease activity but lacks a 3′-5′ proofreading exonuclease activity. When fidelity is required, proofreading exonucleases such as Vent and Deep Vent (New England Biolabs) or Pfu (Stratagene) may be used (Cline et al., (1996) Nucleic Acids Research 24, 3456-3551). In another embodiment of the invention, a single enzyme approach may be used involving a DNA polymerase with intrinsic reverse transcriptase activity, such as Thermus thermophius (Tth) polymerase (Bustin, (2000) Journal of Molecular Endocrinology 25, 169-193. A skilled artisan may appreciate the variety of enzymes available for use in the present invention.

The methodologies and control gene primers of the present invention may be used, for example, in any kinetic RT-PCR methodology, including those that combine fluorescence techniques with instrumentation capable of combining amplification, detection and quantification (Orlando et al., (1998) Clinical Chemistry and Laboratory Medicine 36, 255-269). The choice of instrumentation is particularly important in multiplex RT-PCR, wherein multiple primer sets are used to amplify multiple specific targets simultaneously. This requires simultaneous detection of multiple fluorescent dyes. Accurate quantitation while maintaining a broad dynamic range of sensitivity across mRNA levels is the focus of upcoming technologies, any of which are applicable for use in the present invention. Preferred instrumentation may be selected from the ABI Prism 7700 (Perkin-Elmer-Applied Biosystems), the Lightcycler (Roche Molecular Biochemicals) and iCycler Thermal Cycler. Featured aspects of these products include high-throughput capacities or unique photodetection devices.

Without further description, it is believed that one of ordinary skill in the art can, using the preceding description and the following illustrative examples, practice the methods and use the control genes of the present invention. The following working examples therefore, specifically point out the preferred embodiments of the present invention, and are not to be construed as limiting in any way the remainder of the disclosure.

### EXAMPLES

### Example 1

### Selection of Control Genes

The control genes were selected by querying the Gene Logic GeneExpress® database to create expression profiles from a variety of human cell and tissue samples. Table 3 A-B lists and describes the tissue or cell samples used to identify control genes listed in Tables 1 and 2. The first column of Table 3 identifies the organ of the particular sample, the second details the morphology, and the third column provides the number of samples. Table 3 A-B includes 695 diseased and 560 normal samples.

The GeneExpress® database was produced from data derived from screening various cell or tissue samples using the Affymetrix human chip set. In general, tissue and cell samples were processed following the Affymetrix GeneChip® Expression Analysis Manual. Frozen tissue was first ground to powder using the Spex Certiprep 6800 Freezer Mill. Total RNA was then extracted using Trizol (Invitrogen Life Technologies) followed by a cleanup step utilizing the RNeasy Mini Kit and if required ethanol precipitated to achieve a concentration of 1 μg/pl. Using 10-40 μg of total RNA, double stranded cDNA was created using the SuperScript Choice system (Invitrogen Life Technologies). First strand cDNA synthesis was primed with a T7-(dT₂₄) oligonucleotide. The cDNA was then phenol-chloroform extracted and ethanol precipitated to a final concentration of 1 μg/μl.

55 μg of fragmented cRNA was hybridized on the Human Genome U95 set for twenty-four hours at 60 rpm in a 45° C. hybridization oven, according to the Affymetrix protocol. The chips were washed and stained with Streptavidin Phycoerythrin (SAPE) (Molecular Probes) in Affymetrix fluidics stations. To amplify staining, the chips were washed with SAPE solution, stained with an anti-streptavidin biotinylated antibody (Vector Laboratories) followed by washing with SAPE solution. Hybridization to the probe arrays was detected by fluorometric scanning (Hewlett Packard Gene Array Scanner). Following hybridization and scanning, the microarray images were analyzed for quality control, looking for major chip defects or abnormalities in hybridization signal. After all chips passed quality control, the data was analyzed using Affymetrix GeneChip® software (v3.0).

Gene expression data was then analyzed to identify those genes that are consistently expressed across 1255 normal and disease samples, e.g. being called Present more than 95% of the time. Table 1 provides an initial list of approximately 560 genes with a % CV less than 30% across the normal and disease samples studied. Table 1 also provides the mean expression value, an exemplary GenBank accession number for each of the genes and the standard deviation value from the mean for each gene. The GenBank accession numbers can be used to locate the publicly available sequences and all GenBank accession numbers herein reported at specifically incorporated by reference in their entirety. This list of 560 genes from 1255 normal and diseased samples had been scanned on Affymetrix human U95 A GeneChip® scanned on a high photomultiplier tube (PMT) settings.

The gene list of Table 1 was then re-examined by utilizing human samples run on the Affyymetrix human U95 A GeneChip® scanned on a low photomultiplier tube (PMT) settings. The human samples consisted of 55 human tissue samples and 46 human cancer cell lines. For each of these samples, the mean average difference, standard deviation and % CV were determined for each Affymetrix fragment on the human U95 A GeneChip®. The data was sorted by % CV and those gene fragments with values less than 40% were chosen for fiuther analysis after all genes with underscore annotations were deleted (i.e. _f, _s, _r, etc.) [see www.affimetrix.com].

The high PMT list was then compared with the low PMT list and all genes that were not present on both lists were removed. All genes with underscore annotations were then deleted from the list (i.e. _f, _s, _r, etc.). This resulted in a list of 771 genes. The list was then filtered to show CV values equal or less than 28% at low PMT settings as well as CV values equal or less than 31% at high PMT settings. Six additional human genes with CV values equal or less 37% at low PMT settings and equal or less than 32% at high PMT settings were added to the list. These six genes have rat homologue genes that exhibited constant gene expression over untreated and toxin treated rat samples scanned at low PMT settings (˜200 samples). The resulting control gene list is in Table 2.

### Example 2

### Quantitative PCR Analysis of Expression Levels Using the Control Genes

The expression levels of one or more genes listed in Tables 1 and 2 may be used to normalize gene expression data produced using Quantitative PCR analysis. For example, Table 4 provides sequences for use as Taqman probes along with the forward and reverse primers for three genes: sorting nexin 3, polymerase (RNA) II (DNA directed) polypeptide F, and seryl-tRNA synthetase in Table 1 or 2. Real time PCR detection may be accomplished by the use of the ABI PRISM 7700 Sequence Detection System. The 7700 measures the fluorescence intensity of the sample each cycle and is able to detect the presence of specific amplicons within the PCR reaction. TaqMan® assay provided by Perkin Ebner may be used to assay quantities of RNA. The primers may be designed from each of the identified genes of Table 1 using Primer Express, a program developed by PE to efficiently find primers and probes for specific sequences. These primers may be used in conjunction with SYBR green (Molecular Probes), a nonspecific double stranded DNA dye, to measure the expression level mRNA corresponding to the expression levels of each gene. This gene expression data may then be used to normalize gene expression data of other test genes.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

TABLE 1

| | | | Expression value | | |
|---|---|---|---|---|---|
| | | | Mean | Std_dev | |
| Fragment No. | Gene Name | GenBank No. | (overall) | (overall) | CV % |
| 41785_at | eukaryotic translation initiation factor 4 gamma\| 2 | U73824 | 1825.59 | 392.92 | 21.52 |
| 34392_s_at | RAB1\| member RAS oncogene family | AL050268 | 1493.57 | 315.17 | 21.10 |
| 41194_at | signal recognition particle 14 kD (homologous Alu RNA-binding | AI525652 | 1472.21 | 312.41 | 21.22 |
| | protein) | | | | |
| 41185_f_at | SMT3 (suppressor of mif two 3\| yeast) homolog 2 | AI971724 | 1422.18 | 305.74 | 21.50 |
| 41833_at | jumping translocation breakpoint | AB016492 | 1199.41 | 259.92 | 21.67 |
| 1394_at | ras homolog gene family\| member A | L25080 | 1031.88 | 223.38 | 21.65 |
| 38974_at | RNA-binding protein regulatory subunit | AF021819 | 910.49 | 199.88 | 21.95 |
| 505_at | CDC37 (cell division cycle 37\| S. cerevisiae\| homolog) | U43077 | 628.94 | 137.46 | 21.86 |
| 34849_at | seryl-tRNA synthetase | X91257 | 460.23 | 99.39 | 21.60 |
| 36942_at | KIAA0174 gene product | D79996 | 447.67 | 97.97 | 21.88 |
| 39811_at | hypothetical protein MGC2749 | AA402538 | 400.76 | 85.04 | 21.22 |
| 36110_at | RAB5A\| member RAS oncogene family | M28215 | 389.74 | 82.31 | 21.12 |
| 40819_at | RNA binding motif protein 8A | AA161065 | 372.82 | 81.38 | 21.83 |
| 38672_at | protein phosphatase 1\| regulatory subunit 10 | Y13247 | 249.96 | 53.96 | 21.59 |
| 33778_at | chromosome 22 open reading frame 4 | AL096779 | 223.74 | 48.47 | 21.66 |
| 911_s_at | calmodulin 2 (phosphorylase kinase\| delta) | M19311 | 2123.80 | 478.30 | 22.52 |
| 37448_s_at | guanine nucleotide binding protein (G protein)\| alpha stimulating | X56009 | 1544.74 | 354.90 | 22.97 |
| | activity polypeptide 1\|neuroendocrine secretory protein 55 | | | | |
| 40864_at | ras-related C3 botulinum toxin substrate 1 (rho family\| small GTP | D25274 | 1068.49 | 239.65 | 22.43 |
| | binding protein Rac1) | | | | |
| 41187_at | death-associated protein 6 | U26162 | 1052.43 | 238.95 | 22.70 |
| 39360_at | sorting nexin 3 | AF034546 | 944.20 | 210.75 | 22.32 |
| 32175_at | CDC10 (cell division cycle 10\| S. cerevisiae\| homolog) | S72008 | 878.75 | 197.97 | 22.53 |
| 32145_at | adducin 1 (alpha) | X58141 | 870.17 | 198.24 | 22.78 |
| 38831_f_at | guanine nucleotide binding protein (G protein)\| beta polypeptide 2 | AF053356 | 819.87 | 186.42 | 22.74 |
| 32766_at | thyroid autoantigen 70 kD (Ku antigen) | Z83840 | 804.64 | 180.45 | 22.43 |
| 35753_at | U5 snRNP-specific protein (220 kD)\| ortholog of S. cerevisiae Prp8p | AB007510 | 704.30 | 156.01 | 22.15 |
| 36027_at | polymerase (RNA) II (DNA directed) polypeptide F | AA418779 | 691.92 | 158.65 | 22.93 |
| 38738_at | SMT3 (suppressor of mif two 3\| yeast) homolog 1 | X99584 | 642.57 | 144.63 | 22.51 |
| 38720_at | chaperonin containing TCP1\| subunit 7 (eta) | AF026292 | 642.25 | 141.43 | 22.02 |
| 1695_at | neural precursor cell expressed\| developmentally down-regulated 8 | D23662 | 620.09 | 141.48 | 22.82 |
| 38483_at | hypothetical protein | AJ011916 | 605.91 | 135.19 | 22.31 |
| 38758_at | PDGFA associated protein 1 | R98910 | 593.33 | 135.20 | 22.79 |
| 39782_at | nuclear DNA-binding protein | X95592 | 539.50 | 118.97 | 22.05 |
| 41132_r_at | heterogeneous nuclear ribonucleoprotein H2 (H′) | U01923 | 537.58 | 120.94 | 22.50 |
| 34864_at | hypothetical protein | AF070638 | 528.37 | 117.67 | 22.27 |
| 35835_at | anaphase-promoting complex subunit 5\| periodontal ligament | AB019409 | 506.73 | 112.50 | 22.20 |
| | fibroblast protein | | | | |
| 32575_at | nucleosome assembly protein 1-like 4 | U77456 | 466.80 | 103.15 | 22.10 |
| 38801_at | VAMP (vesicle-associated membrane protein)-associated protein A | AI742846 | 451.66 | 101.60 | 22.50 |
| | (33 kD) | | | | |
| 36611_at | acid phosphatase 1\| soluble | U25849 | 439.95 | 97.31 | 22.12 |
| 31826_at | KIAA0674 protein | AB014574 | 437.28 | 99.45 | 22.74 |
| 39868_at | poly(rC)-binding protein 3 | AL046394 | 367.96 | 84.27 | 22.90 |
| 40824_at | RAN binding protein 16 | AB018288 | 340.15 | 77.74 | 22.85 |
| 33826_at | Cip1-interacting zinc finger protein | AL120500 | 332.11 | 73.81 | 22.23 |
| 37362_at | RAB5B\| member RAS oncogene family | X54871 | 269.39 | 59.71 | 22.16 |
| 40836_s_at | metastasis-associated 1-like 1 | W26677 | 217.72 | 47.91 | 22.01 |
| 32820_at | CCR4-NOT transcription complex\| subunit 4 | U71267 | 210.33 | 46.91 | 22.30 |
| 35850_at | phosphatidylserine receptor | AI950382 | 173.78 | 39.77 | 22.88 |
| 39136_at | oxidative-stress responsive 1 | AB017642 | 170.33 | 39.12 | 22.97 |
| 41276_at | sin3-associated polypeptide\| 18 kD | W27641 | 140.38 | 31.56 | 22.48 |
| 36702_at | T-box 19 | AJ010277 | 135.27 | 31.04 | 22.94 |
| 37309_at | ras homolog gene family\| member A | L09159 | 2015.36 | 468.39 | 23.24 |
| 39740_g_at | nascent-polypeptide-associated complex alpha polypeptide | AF054187 | 1774.49 | 419.00 | 23.61 |
| 35746_r_at | poly(rC)-binding protein 2 | X78136 | 1615.54 | 383.34 | 23.73 |
| 39758_f_at | lysosomal-associated membrane protein 1 | J04182 | 1519.81 | 364.27 | 23.97 |
| 41221_at | phosphoglycerate mutase 1 (brain) | J04173 | 1508.34 | 355.87 | 23.59 |
| 1420_s_at | eukaryotic translation initiation factor 4A\| isoform 2 | D30655 | 1435.89 | 338.77 | 23.59 |
| 39415_at | heterogeneous nuclear ribonucleoprotein K | X72727 | 1361.71 | 317.49 | 23.32 |
| 40125_at | calnexin | L10284 | 1244.21 | 293.89 | 23.62 |
| 32590_at | nucleolin | M60858 | 1156.35 | 266.25 | 23.02 |
| 36972_at | coated vesicle membrane protein | X92098 | 1148.13 | 268.13 | 23.35 |
| 35307_at | GDP dissociation inhibitor 2 | Y13286 | 1002.33 | 230.95 | 23.04 |
| 324_f_at | EST | HG1515-HT1515 | 1001.35 | 232.80 | 23.25 |
| 880_at | FK506-binding protein 1A (12 kD) | M34539 | 887.82 | 205.06 | 23.10 |
| 41295_at | GTT1 protein | AL041780 | 878.34 | 203.61 | 23.18 |
| 37040_at | KIAA0088 protein | D42041 | 826.78 | 194.69 | 23.55 |
| 723_s_at | heterogeneous nuclear ribonucleoprotein C (C1/C2) | HG1322-HT5143 | 744.87 | 172.78 | 23.20 |
| 39184_at | transcription elongation factor B (SIII)\| polypeptide 2 (18 kD\| elongin B) | AI857469 | 734.63 | 173.55 | 23.62 |
| 34336_at | lysyl-tRNA synthetase | D32053 | 705.01 | 162.51 | 23.05 |
| 41269_r_at | API5-like 1 | Y15906 | 680.76 | 162.93 | 23.93 |
| 33341_at | guanine nucleotide binding protein (G protein)\| beta polypeptide 1 | X04526 | 650.30 | 150.09 | 23.08 |
| 1311_at | proteasome (prosome\| macropain) subunit\| beta type\| 4 | D26600 | 619.24 | 143.61 | 23.19 |
| 410_s_at | casein kinase 2\| beta polypeptide | X57152 | 613.46 | 141.25 | 23.03 |
| 34402_at | unr-interacting protein | AB024327 | 603.22 | 142.42 | 23.61 |
| 36949_at | casein kinase 1\| delta | U29171 | 546.48 | 130.09 | 23.81 |
| 941_at | proteasome (prosome\| macropain) subunit\| beta type\| 6 | D29012 | 542.60 | 126.93 | 23.39 |
| 33397_at | CDP-diacylglycerol-inositol 3-phosphatidyltransferase | AL050383 | 480.14 | 113.09 | 23.55 |
| | (phosphatidylinositol synthase) | | | | |
| 39778_at | mannosyl (alpha-1\|3-)-glycoprotein beta-1\|2-N- | M55621 | 474.90 | 111.56 | 23.49 |
| | acetylglucosaminyltransferase | | | | |
| 38710_at | hypothetical protein FLJ20113 | AL096714 | 456.86 | 105.09 | 23.00 |
| 33215_g_at | mitochondrial ribosomal protein S12 | Y11681 | 439.32 | 103.28 | 23.51 |
| 33388_at | EST | AL080223 | 436.00 | 102.58 | 23.53 |
| 38016_at | heterogeneous nuclear ribonucleoprotein D (AU-rich element RNA- | M94630 | 409.89 | 96.46 | 23.53 |
| | binding protein 1\| 37 kD) | | | | |
| 632_at | glycogen synthase kinase 3 alpha | L40027 | 373.13 | 88.22 | 23.64 |
| 34346_at | protein kinase\| AMP-activated\| gamma 1 non-catalytic subunit | U42412 | 362.28 | 83.94 | 23.17 |
| 41597_s_at | SEC22\| vesicle trafficking protein (S. cerevisiae)-like 1 | AF047442 | 307.93 | 72.68 | 23.60 |
| 1874_at | RAD23 (S. cerevisiae) homolog B | D21090 | 292.62 | 69.19 | 23.64 |
| 39047_at | KIAA0156 gene product | AB020880 | 277.73 | 66.29 | 23.87 |
| 36574_at | isocitrate dehydrogenase 3 (NAD+) gamma | Z68907 | 275.63 | 63.80 | 23.15 |
| 39164_at | ariadne (Drosophila) homolog 2 | AF099149 | 225.58 | 52.80 | 23.41 |
| 41727_at | KIAA1007 protein | AB023224 | 153.39 | 36.64 | 23.89 |
| 41483_s_at | jun D proto-oncogene | X56681 | 2091.94 | 520.54 | 24.88 |
| 38542_at | nucleophosmin (nucleolar phosphoprotein B23\| numatrin) | U89322 | 1801.84 | 448.04 | 24.87 |
| 1180_g_at | heat shock 70 kD protein 8 | HG2855-HT2995 | 1774.59 | 428.21 | 24.13 |
| 35055_at | basic transcription factor 3 | X53281 | 1657.93 | 403.15 | 24.32 |
| 254_at | H3 histone\| family 3A | M11353 | 1590.81 | 382.95 | 24.07 |
| 32316_s_at | heat shock 90 kD protein 1\| alpha | X15183 | 1482.06 | 365.51 | 24.66 |
| 39025_at | 6.2 kd protein | AI557912 | 1345.64 | 334.33 | 24.85 |
| 33458_r_at | H2B histone family\| member L | AI688098 | 1220.62 | 297.73 | 24.39 |
| 36994_at | ATPase\| H+ transporting\| lysosomal (vacuolar proton pump) 16 kD | M62762 | 1072.06 | 262.59 | 24.49 |
| 409_at | tyrosine 3-monooxygenase/tryptophan 5-monooxygenase activation | X56468 | 983.68 | 241.73 | 24.57 |
| | protein\| theta polypeptide | | | | |
| 33987_at | ADP-ribosylation factor 1 | M36340 | 968.58 | 233.00 | 24.06 |
| 1268_at | ubiquitin-activating enzyme E1 (A1S9T and BN75 temperature | M58028 | 897.16 | 219.32 | 24.45 |
| | sensitivity complementing) | | | | |
| 37012_at | capping protein (actin filament) muscle Z-line\| beta | U03271 | 812.51 | 195.70 | 24.09 |
| 39030_at | Rab acceptor 1 (prenylated) | AJ133534 | 800.58 | 194.20 | 24.26 |
| 39866_at | ubiquitin specific protease 22 | AB028986 | 789.47 | 194.01 | 24.57 |
| 36186_at | RNA-binding protein S1\| serine-rich domain | L37368 | 766.92 | 190.58 | 24.85 |
| 35754_at | EST | L40391 | 740.40 | 178.58 | 24.12 |
| 33666_at | heterogeneous nuclear ribonucleoprotein C (C1/C2) | M16342 | 683.81 | 170.55 | 24.94 |
| 36517_at | U2(RNU2) small nuclear RNA auxillary factor 1 (non-standard | M96982 | 666.76 | 165.25 | 24.78 |
| | symbol) | | | | |
| 40189_at | SET translocation (myeloid leukemia-associated) | M93651 | 658.30 | 158.80 | 24.12 |
| 33875_at | ATPase\| H+ transporting\| lysosomal (vacuolar proton pump) 9 kD | AI547262 | 656.69 | 162.39 | 24.73 |
| 41224_at | KIAA0788 protein | AB018331 | 622.29 | 154.12 | 24.77 |
| 41241_at | asparaginyl-tRNA synthetase | D84273 | 608.42 | 151.24 | 24.86 |
| 38413_at | defender against cell death 1 | D15057 | 554.80 | 136.87 | 24.67 |
| 33198_at | binder of Arl Two | AA206524 | 544.09 | 135.81 | 24.96 |
| 41309_g_at | C-terminal binding protein 1 | U37408 | 537.50 | 132.44 | 24.64 |
| 1295_at | v-rel avian reticuloendotheliosis viral oncogene homolog A (nuclear | L19067 | 527.61 | 127.46 | 24.16 |
| | factor of kappa light polypeptide gene enhancer in B-cells 3 (p65)) | | | | |
| 41830_at | KIAA0494 gene product | AB007983 | 511.59 | 124.66 | 24.37 |
| 32241_at | TAR DNA binding protein | AL050265 | 510.14 | 127.08 | 24.91 |
| 34330_at | cytochrome c oxidase subunit VIIa polypeptide 2 like | AB007618 | 499.29 | 121.64 | 24.36 |
| 41737_at | Ser/Arg-related nuclear matrix protein (plenty of prolines 101-like) | AF048977 | 488.41 | 118.20 | 24.20 |
| 38297_at | phosphatidylinositol transfer protein\| membrane-associated | X98654 | 463.69 | 112.50 | 24.26 |
| 457_s_at | ubiquitin-like 1 (sentrin) | U67122 | 452.05 | 109.04 | 24.12 |
| 32832_at | macrophage erythroblast attacher | AF084928 | 413.07 | 100.17 | 24.25 |
| 39147_g_at | alpha thalassemia/mental retardation syndrome X-linked (RAD54 (S. | U72936 | 385.04 | 95.45 | 24.79 |
| | cerevisiae) homolog) | | | | |
| 38659_at | suppressor of clear\| C. elegans\| homolog of | AB020669 | 341.88 | 84.08 | 24.59 |
| 40988_at | YME1 (S. cerevisiae)-like 1 | AJ132637 | 341.77 | 82.87 | 24.25 |
| 38412_at | protein phosphatase 1\| regulatory (inhibitor) subunit 11 | U53588 | 336.42 | 81.37 | 24.19 |
| 35790_at | vacuolar protein sorting 26 (yeast homolog) | AF054179 | 328.77 | 80.27 | 24.42 |
| 35534_at | KIAA0514 gene product | AB011086 | 314.36 | 77.83 | 24.76 |
| 36019_at | serine/threonine kinase 19 | L26260 | 310.58 | 76.38 | 24.59 |
| 40130_at | follistatin-like 1\|hypothetical protein FLJ22169 | U06863 | 298.89 | 72.78 | 24.35 |
| 34906_g_at | glutamate receptor, ionotropic, kainate 5 | AA977136 | 268.60 | 66.46 | 24.74 |
| 40404_s_at | CDC16 (cell division cycle 16\| S. cerevisiae\| homolog) | U18291 | 256.93 | 63.06 | 24.54 |
| 40426_at | B-cell CLL/lymphoma 7B | X89985 | 244.40 | 60.44 | 24.73 |
| 41540_at | protein phosphatase 1\| regulatory subunit 7 | Z50749 | 240.53 | 59.73 | 24.83 |
| 34231_at | histone acetyltransferase | AF074606 | 237.47 | 59.16 | 24.91 |
| 36166_at | splicing factor similar to dnaJ | AF083190 | 226.95 | 55.72 | 24.55 |
| 36579_at | ubiquitination factor E4A (homologous to yeast UFD2) | D50916 | 225.21 | 56.06 | 24.89 |
| 1843_at | EST | HG2825-HT2949 | 220.98 | 55.23 | 24.99 |
| 41374_at | ribosomal protein S6 kinase\| 70 kD\| polypeptide 2 | AB016869 | 217.24 | 54.19 | 24.94 |
| 33394_at | hypothetical protein FLJ11126 | AA034074 | 212.62 | 51.45 | 24.20 |
| 40816_at | nuclear phosphoprotein similar to S. cerevisiae PWP1 | L07758 | 212.08 | 51.86 | 24.45 |
| 36003_at | poly(A)-specific ribonuclease (deadenylation nuclease) | AJ005698 | 197.97 | 47.83 | 24.16 |
| 147_at | tumor susceptibility gene 101 | U82130 | 190.90 | 47.73 | 25.00 |
| 32234_at | dystonia 1\| torsion (autosomal dominant; torsin A) | AF007871 | 172.16 | 41.43 | 24.07 |
| 1612_s_at | jun D proto-oncogene | X56681 | 2085.72 | 539.45 | 25.86 |
| 36587_at | eukaryotic translation elongation factor 2 | Z11692 | 1910.61 | 487.20 | 25.50 |
| 31952_at | ribosomal protein L6 | X69391 | 1813.14 | 468.79 | 25.86 |
| 33820_g_at | lactate dehydrogenase B | X13794 | 1781.14 | 453.34 | 25.45 |
| 31573_at | ribosomal protein S25 | M64716 | 1699.81 | 438.15 | 25.78 |
| 31955_at | Finkel-Biskis-Reilly murine sarcoma virus (FBR-MuSV) ubiquitously | X65923 | 1620.25 | 408.37 | 25.20 |
| | expressed (fox derived); ribosomal protein S30 | | | | |
| 33943_at | ferritin\| heavy polypeptide 1 | L20941 | 1570.65 | 402.53 | 25.63 |
| 34891_at | dynein\| cytoplasmic\| light polypeptide | AI540958 | 1557.07 | 397.55 | 25.53 |
| 34646_at | ribosomal protein S7 | Z25749 | 1458.01 | 366.55 | 25.14 |
| 33451_s_at | ribosomal protein L22 | AI526079 | 1318.46 | 331.97 | 25.18 |
| 39019_at | lysosomal-associated protein transmembrane 4 alpha | D14696 | 1285.43 | 328.63 | 25.57 |
| 40281_at | neural precursor cell expressed\| developmentally down-regulated 5 | D63878 | 1159.40 | 298.10 | 25.71 |
| 41724_at | accessory proteins BAP31/BAP29 | X81817 | 1130.18 | 287.58 | 25.45 |
| 36986_at | lysophospholipase II | AL031295 | 945.11 | 244.21 | 25.84 |
| 34877_at | Janus kinase 1 (a protein tyrosine kinase) | AL039831 | 920.60 | 236.94 | 25.74 |
| 35787_at | dynein\| cytoplasmic\| intermediate polypeptide 2 | AI986201 | 919.87 | 231.48 | 25.16 |
| 39003_at | pituitary tumor-transforming 1 interacting protein | Z50022 | 915.25 | 235.56 | 25.74 |
| 36654_s_at | heterogeneous nuclear ribonucleoprotein A2/B1 | M29065 | 900.89 | 230.63 | 25.60 |
| 35292_at | HLA-B associated transcript-1 | Z37166 | 815.55 | 210.60 | 25.82 |
| 38657_s_at | clathrin\| light polypeptide (Lca) | M20471 | 804.86 | 208.87 | 25.95 |
| 32220_at | high-mobility group (nonhistone chromosomal) protein 1 | D63874 | 756.62 | 189.42 | 25.04 |
| 38663_at | Breakpoint cluster region protein\| uterine leiomyoma\| 1; barrier to | AI033692 | 730.14 | 188.61 | 25.83 |
| | autointegration factor | | | | |
| 38733_at | X-ray repair complementing defective repair in Chinese hamster cells | M30938 | 699.28 | 175.17 | 25.05 |
| | 5 (double-strand-break rejoining; Ku autoantigen\| 80 kD) | | | | |
| 35749_at | transcriptional adaptor 3 (ADA3\| yeast homolog)-like (PCAF histone | AF069733 | 629.52 | 161.07 | 25.59 |
| | acetylase complex) | | | | |
| 223_at | ubiquitin-conjugating enzyme E2L 3 | S81003 | 585.33 | 148.38 | 25.35 |
| 1499_at | farnesyltransferase\| CAAX box\| alpha | L10413 | 576.15 | 146.04 | 25.35 |
| 35783_at | vesicle-associated membrane protein 3 (cellubrevin) | H93123 | 532.51 | 133.72 | 25.11 |
| 35279_at | Tax1 (human T-cell leukemia virus type I) binding protein 1 | U33821 | 483.39 | 123.00 | 25.45 |
| 40063_at | nuclear domain 10 protein | U22897 | 479.54 | 121.16 | 25.27 |
| 36645_at | v-rel avian reticuloendotheliosis viral oncogene homolog A (nuclear | L19067 | 467.83 | 119.55 | 25.55 |
| | factor of kappa light polypeptide gene enhancer in B-cells 3 (p65)) | | | | |
| 37389_at | small acidic protein | AI346580 | 456.73 | 116.38 | 25.48 |
| 36137_at | chromodomain helicase DNA binding protein 4 | X86691 | 453.01 | 114.59 | 25.30 |
| 32836_at | 1-acylglycerol-3-phosphate O-acyltransferase 1 (lysophosphatidic | U56417 | 452.04 | 116.91 | 25.86 |
| | acid acyltransferase\| alpha) | | | | |
| 38450_at | Sjogren syndrome antigen B (autoantigen La) | X69804 | 433.17 | 110.29 | 25.46 |
| 41366_at | KIAA1002 protein | AB023219 | 392.83 | 101.32 | 25.79 |
| 37321_at | tetratricopeptide repeat domain 1 | U46570 | 378.24 | 97.01 | 25.65 |
| 35359_at | KIAA0235 protein | D87078 | 356.06 | 90.11 | 25.31 |
| 41335_at | DKFZP566O1646 protein | AL050084 | 348.57 | 89.78 | 25.76 |
| 32586_at | KIAA0217 protein | D86971 | 321.19 | 82.43 | 25.67 |
| 32591_at | HCDI protein | AI494623 | 310.71 | 80.25 | 25.83 |
| 35187_at | EST | AL080216 | 307.74 | 77.04 | 25.04 |
| 39435_at | prefoldin 1 | D45333 | 305.60 | 76.71 | 25.10 |
| 40414_at | valyl-tRNA synthetase 2 | X59303 | 303.73 | 77.00 | 25.35 |
| 40469_at | minichromosome maintenance deficient (S. cerevisiae) 3-associated | AB011144 | 292.48 | 73.84 | 25.24 |
| | protein | | | | |
| 36080_at | clock (mouse) homolog | AB002332 | 260.18 | 67.26 | 25.85 |
| 34370_at | archain 1 | X81198 | 250.74 | 63.61 | 25.37 |
| 32039_at | adaptor-related protein complex 3\| beta 1 subunit | U81504 | 233.73 | 59.69 | 25.54 |
| 40849_s_at | cAMP responsive element binding protein 3 (luman) | U88528 | 229.61 | 57.97 | 25.25 |
| 33861_at | CCR4-NOT transcription complex\| subunit 2 | AI123426 | 211.54 | 54.09 | 25.57 |
| 36603_at | GCN1 (general control of amino-acid synthesis 1\| yeast)-like | D86973 | 199.11 | 50.97 | 25.60 |
| | 1\| homeodomain-interacting protein kinase 2 | | | | |
| 40052_at | ARP1 (actin-related protein 1\| yeast) homolog A (centractin alpha) | X82206 | 185.41 | 47.96 | 25.87 |
| 314_at | phosphatidylinositol glycan\| class B | D42138 | 183.52 | 46.89 | 25.55 |
| 31860_at | putative receptor protein | X51804 | 151.68 | 38.76 | 25.56 |
| 32713_at | golgi autoantigen\| golgin subfamily a\| 1 | U51587 | 147.24 | 38.12 | 25.89 |
| 35743_at | cleavage and polyadenylation specific factor 4\| 30 kD subunit | U79569 | 128.76 | 33.05 | 25.67 |
| 31708_at | ribosomal protein L30 | L05095 | 2416.22 | 650.09 | 26.91 |
| 32315_at | ribosomal protein S24 | M31520 | 2358.55 | 632.51 | 26.82 |
| 327_f_at | EST | HG1800-HT1823 | 2348.03 | 625.69 | 26.65 |
| 36333_at | ribosomal protein L7 | X57958 | 2277.08 | 599.76 | 26.34 |
| 33677_at | ribosomal protein L24 | M94314 | 2153.43 | 571.37 | 26.53 |
| 33657_at | ribosomal protein L34 | L38941 | 2146.73 | 566.47 | 26.39 |
| 32487_s_at | karyopherin alpha 4 (importin alpha 3)\|ribosomal protein\| large P2 | AB002533 | 2119.07 | 567.25 | 26.77 |
| 33660_at | ribosomal protein L5 | U14966 | 2010.05 | 527.61 | 26.25 |
| 39830_at | ribosomal protein L27 | AA044823 | 1990.00 | 530.31 | 26.65 |
| 1367_f_at | ubiquitin C | M26880 | 1975.66 | 520.20 | 26.33 |
| 36795_at | prosaposin (variant Gaucher disease and variant metachromatic | J03077 | 1898.29 | 499.48 | 26.31 |
| | leukodystrophy) | | | | |
| 41231_f_at | high-mobility group (nonhistone chromosomal) protein 17 | X13546 | 1850.84 | 490.00 | 26.47 |
| 33984_at | heat shock 90 kD protein 1\| beta | M16660 | 1677.04 | 446.26 | 26.61 |
| 32394_s_at | ribosomal protein L23 | X55954 | 1630.44 | 424.17 | 26.02 |
| 33485_at | ribosomal protein L4 | D23660 | 1623.23 | 432.42 | 26.64 |
| 31907_at | ribosomal protein L14 | D87735 | 1588.57 | 413.70 | 26.04 |
| 32272_at | tubulin\| alpha\| ubiquitous | K00558 | 1519.01 | 408.21 | 26.87 |
| 34381_at | cytochrome c oxidase subunit VIIc | AI708889 | 1483.67 | 395.32 | 26.64 |
| 41256_at | eukaryotic translation elongation factor 1 delta (guanine nucleotide | Z21507 | 1328.54 | 348.13 | 26.20 |
| | exchange protein) | | | | |
| 41768_at | protein kinase\| cAMP-dependent\| regulatory\| type 1\| alpha (tissue | M33336 | 1302.05 | 349.10 | 26.81 |
| | specific extinguisher 1) | | | | |
| 1009_at | histidine triad nucleotide-binding protein | U51004 | 1133.18 | 301.26 | 26.59 |
| 39856_at | ribosomal protein L36a | AI708983 | 1124.95 | 298.25 | 26.51 |
| 36138_at | calpain 4\| small subunit (30K) | X04106 | 1011.52 | 270.95 | 26.79 |
| 40637_at | heat shock 70 kD protein 8 | Y00371 | 996.35 | 262.73 | 26.37 |
| 38527_at | non-POU-domain-containing\| octamer-binding | U02493 | 944.25 | 254.22 | 26.92 |
| 37364_at | B-cell associated protein | U72511 | 894.01 | 235.05 | 26.29 |
| 39800_s_at | HS1 binding protein | U68566 | 816.62 | 213.20 | 26.11 |
| 39336_at | ADP-ribosylation factor 3 | M74491 | 717.23 | 189.84 | 26.47 |
| 32530_at | tyrosine 3-monooxygenase/tryptophan 5-monooxygenase activation | X56468 | 698.12 | 184.17 | 26.38 |
| | protein\| theta polypeptide | | | | |
| 905_at | guanylate kinase 1 | L76200 | 678.74 | 181.25 | 26.70 |
| 838_s_at | ubiquitin-conjugating enzyme E2I (homologous to yeast UBC9) | U45328 | 662.09 | 176.95 | 26.73 |
| 33154_at | proteasome (prosome\| macropain) subunit\| beta type\| 4 | D26600 | 658.73 | 174.10 | 26.43 |
| 688_at | proteasome (prosome\| macropain) 26S subunit\| ATPase\| 1 | L02426 | 651.35 | 169.84 | 26.07 |
| 35316_at | Ras-related GTP-binding protein | U41654 | 634.52 | 171.30 | 27.00 |
| 31906_at | heat shock factor binding protein 1 | AF068754 | 632.01 | 166.68 | 26.37 |
| 1450_g_at | proteasome (prosome\| macropain) subunit\| alpha type\| 4 | D00763 | 602.83 | 160.76 | 26.67 |
| 1641_s_at | damage-specific DNA binding protein 1 (127 kD) | U32986 | 585.59 | 155.10 | 26.49 |
| 39079_at | enhancer of rudimentary (Drosophila) homolog | D85758 | 574.87 | 154.61 | 26.89 |
| 35302_at | nuclear RNA export factor 1 | AJ132712 | 530.82 | 139.38 | 26.26 |
| 37395_at | ATPase\| vacuolar\| 14 kD | D49400 | 521.98 | 136.38 | 26.13 |
| 1158_s_at | calmodulin 3 (phosphorylase kinase\| delta) | J04046 | 515.17 | 138.95 | 26.97 |
| 39346_at | GAP-associated tyrosine phosphoprotein p62 (Sam68) | M88108 | 506.62 | 133.58 | 26.37 |
| 868_at | TATA box binding protein (TBP)-associated factor\| RNA polymerase | U13991 | 495.16 | 131.44 | 26.54 |
| | II\| H\| 30 kD | | | | |
| 37367_at | ATPase\| H+ transporting\| lysosomal (vacuolar proton pump) 31 kD | X76228 | 491.45 | 130.10 | 26.47 |
| 498_at | Tax1 (human T-cell leukemia virus type I) binding protein 1 | U33821 | 482.31 | 128.42 | 26.63 |
| 36571_at | topoisomerase (DNA) II beta (180 kD) | X68060 | 471.68 | 123.52 | 26.19 |
| 37719_at | myeloid leukemia factor 2 | AF070539 | 468.02 | 123.95 | 26.48 |
| 35337_at | F-box only protein 7 | AL050254 | 442.72 | 116.37 | 26.28 |
| 41170_at | KIAA0663 gene product | AB014563 | 406.22 | 106.10 | 26.12 |
| 33818_at | valosin-containing protein | AC004472 | 396.96 | 104.80 | 26.40 |
| 34773_at | tubulin-specific chaperone a | AF038952 | 374.99 | 100.24 | 26.73 |
| 41413_at | cleft lip and palate associated transmembrane protein 1 | AF037339 | 362.49 | 94.27 | 26.01 |
| 37336_at | UBX domain-containing 1 | D87684 | 337.75 | 89.52 | 26.51 |
| 35826_at | suppressor of Ty (S. cerevisiae) 5 homolog | AF040253 | 335.01 | 89.78 | 26.80 |
| 37031_at | C9orf10 protein | D80005 | 332.88 | 89.07 | 26.76 |
| 37010_at | general transcription factor IIA\| 2 (12 kD subunit) | AI203737 | 330.68 | 87.21 | 26.37 |
| 40048_at | KIAA0099 gene product | D43951 | 313.60 | 84.42 | 26.92 |
| 41606_at | developmentally regulated GTP-binding protein 1 | AJ005940 | 310.48 | 83.31 | 26.83 |
| 34089_at | KIAA1030 protein | AB028953 | 305.13 | 80.75 | 26.46 |
| 155_s_at | ubiquitin-like 1 (sentrin) | U61397 | 301.59 | 78.76 | 26.12 |
| 37928_at | nuclear transcription factor Y\| beta | AA621555 | 294.85 | 76.93 | 26.09 |
| 1119_at | replication protein A2 (32 kD) | J05249 | 287.23 | 76.53 | 26.65 |
| 38809_s_at | exostoses (multiple)-like 3 | AB011091 | 284.07 | 74.72 | 26.30 |
| 35750_at | uncharacterized hypothalamus protein HT010 | AL049948 | 281.29 | 73.86 | 26.26 |
| 39723_at | cullin 1 | AF062536 | 266.89 | 71.32 | 26.72 |
| 32171_at | eukaryotic translation initiation factor 5 | AL080102 | 264.68 | 69.05 | 26.09 |
| 35353_at | proteasome (prosome\| macropain) 26S subunit\| ATPase\| 2 | D11094 | 261.18 | 69.75 | 26.71 |
| 41151_at | SKIP for skeletal muscle and kidney enriched inositol phosphatase | U45973 | 254.76 | 68.53 | 26.90 |
| 452_at | SWI/SNF related\| matrix associated\| actin dependent regulator of | U66615 | 251.78 | 66.11 | 26.26 |
| | chromatin\| subfamily c\| member 1 | | | | |
| 40160_at | KIAA0618 gene product | AL080109 | 251.72 | 66.68 | 26.49 |
| 38093_at | CGI-35 protein | U90909 | 250.92 | 67.43 | 26.87 |
| 32658_at | SAC2 suppressor of actin mutations 2-like (yeast) | AL031228 | 248.89 | 66.05 | 26.54 |
| 37737_at | protein-L-isoaspartate (D-aspartate) O-methyltransferase | D25547 | 241.91 | 65.11 | 26.91 |
| 32592_at | KIAA0323 protein | AB002321 | 239.55 | 64.28 | 26.83 |
| 35244_at | KIAA0460 protein | AB007929 | 200.99 | 53.83 | 26.78 |
| 37390_at | pre-mRNA splicing factor similar to S. cerevisiae Prp16 | D86977 | 176.59 | 47.50 | 26.90 |
| 1711_at | tumor protein p53-binding protein\| 1 | U09477 | 166.58 | 43.59 | 26.17 |
| 37609_at | nucleotide binding protein 1 (E. coli MinD like) | U01833 | 116.31 | 31.25 | 26.87 |
| 35125_at | ribosomal protein S6 | X67309 | 2371.33 | 654.78 | 27.61 |
| 1676_s_at | eukaryotic translation elongation factor 1 gamma | M55409 | 2315.63 | 639.50 | 27.62 |
| 1653_at | ribosomal protein S3A | M84711 | 2275.89 | 629.87 | 27.68 |
| 1323_at | ubiquitin B | X04803 | 2199.84 | 599.98 | 27.27 |
| 32153_s_at | ubiquitin B | U49869 | 2155.85 | 584.70 | 27.12 |
| 34644_at | beta-2-microglobulin | AB021288 | 2144.72 | 595.49 | 27.77 |
| 41206_r_at | cytochrome c oxidase subunit VIa polypeptide 1 | AI540925 | 2133.29 | 582.59 | 27.31 |
| 32440_at | ribosomal protein L17 | X53777 | 2071.48 | 570.77 | 27.55 |
| 39739_at | nascent-polypeptide-associated complex alpha polypeptide | AF054187 | 2019.90 | 554.73 | 27.46 |
| 34570_at | ribosomal protein S27a | S79522 | 1941.41 | 542.32 | 27.93 |
| 31538_at | ribosomal protein\| large\| P0 | M17885 | 1935.63 | 541.28 | 27.96 |
| 37450_r_at | guanine nucleotide binding protein (G protein)\| alpha stimulating | X04409 | 1931.64 | 523.01 | 27.08 |
| | activity polypeptide 1\|neuroendocrine secretory protein 55 | | | | |
| 31509_at | ribosomal protein L13 | X64707 | 1893.41 | 517.78 | 27.35 |
| 32337_at | ribosomal protein L21 (gene or pseudogene) | U25789 | 1824.69 | 495.86 | 27.17 |
| 41152_f_at | ribosomal protein L44 | T89651 | 1734.56 | 485.33 | 27.98 |
| 34609_g_at | guanine nucleotide binding protein (G protein)\| beta polypeptide 2-like 1 | M24194 | 1733.33 | 479.51 | 27.66 |
| 37677_at | phosphoglycerate kinase 1 | V00572 | 1525.89 | 417.32 | 27.35 |
| 1836_at | cyclin\| | D50310 | 1498.70 | 412.79 | 27.54 |
| 33619_at | ribosomal protein S13 | L01124 | 1395.71 | 390.29 | 27.96 |
| 39738_at | EST | Z82215 | 1392.97 | 382.38 | 27.45 |
| 41213_at | peroxiredoxin 1 | X67951 | 1368.15 | 380.40 | 27.80 |
| 41765_at | ribosomal protein L35 | AI541285 | 1334.55 | 364.70 | 27.33 |
| 37720_at | heat shock 60 kD protein 1 (chaperonin) | M22382 | 1265.49 | 353.60 | 27.94 |
| 1179_at | EST | HG2855-HT2995 | 1261.82 | 345.61 | 27.39 |
| 35745_f_at | poly(rC)-binding protein 2 | X78136 | 1208.03 | 333.10 | 27.57 |
| 40436_g_at | solute carrier family 25 (mitochondrial carrier; adenine nucleotide | J03592 | 1074.34 | 291.35 | 27.12 |
| | translocator)\| member 6 | | | | |
| 970_r_at | ubiquitin specific protease 9\| X chromosome (Drosophila fat facets | X98296 | 953.52 | 264.06 | 27.69 |
| | related) | | | | |
| 35363_at | DEAD/H (Asp-Glu-Ala-Asp/His) box polypeptide 17 (72 kD) | AL080113 | 946.56 | 263.90 | 27.88 |
| 36147_at | signal sequence receptor\| beta (translocon-associated protein beta) | X74104 | 836.90 | 233.12 | 27.86 |
| 35298_at | eukaryotic translation initiation factor 3\| subunit 7 (zeta\| 66/67 kD) | U54558 | 826.39 | 230.28 | 27.87 |
| 39127_f_at | protein phosphatase 2A\| regulatory subunit B′ (PR 53) | X73478 | 817.98 | 228.78 | 27.97 |
| 38084_at | chromobox homolog 3 (Drosophila HP1 gamma) | AA648295 | 809.23 | 222.77 | 27.53 |
| 36111_s_at | hypothetical protein ET\| splicing factor\| arginine/serine-rich 2 | X75755 | 779.82 | 210.62 | 27.01 |
| 38110_at | syndecan binding protein (syntenin) | AF000652 | 761.28 | 212.10 | 27.86 |
| 36928_at | zinc finger protein 146 | X70394 | 728.32 | 198.65 | 27.27 |
| 32573_at | splicing factor\| arginine/serine-rich 9 | AL021546 | 722.59 | 200.04 | 27.68 |
| 162_at | ubiquitin specific protease 11 | U44839 | 717.68 | 194.67 | 27.13 |
| 36152_at | GDP dissociation inhibitor 1 | X79353 | 681.78 | 187.98 | 27.57 |
| 36981_at | signal recognition particle 9 kD | AF070649 | 681.11 | 186.36 | 27.36 |
| 35336_at | EST | AL021707 | 628.65 | 174.85 | 27.81 |
| 38736_at | WD repeat domain 1 | AL050108 | 608.50 | 165.27 | 27.16 |
| 37346_at | ADP-ribosylation factor 5 | M57567 | 600.57 | 167.03 | 27.81 |
| 36991_at | splicing factor\| arginine/serine-rich 4 | L14076 | 565.58 | 156.93 | 27.75 |
| 34393_r_at | RAB1\| member RAS oncogene family | AL050268 | 527.91 | 142.62 | 27.02 |
| 36187_at | ribonuclease/angiogenin inhibitor | X13973 | 520.02 | 143.17 | 27.53 |
| 36035_at | anchor attachment protein 1 (Gaa1p\| yeast) homolog | AB002135 | 518.45 | 142.81 | 27.55 |
| 31893_at | ADP-ribosylation factor-like 2 | L13687 | 511.24 | 140.35 | 27.45 |
| 37730_at | EBNA-2 co-activator (100 kD) | U22055 | 499.00 | 137.76 | 27.61 |
| 37717_at | heterogeneous nuclear ribonucleoprotein M | L03532 | 489.66 | 135.30 | 27.63 |
| 40791_at | polymerase (RNA) II (DNA directed) polypeptide A (220 kD) | X63564 | 487.70 | 135.64 | 27.81 |
| 38990_at | F-box only protein 9 | AL031178 | 483.87 | 130.86 | 27.04 |
| 37666_at | proteasome (prosome\| macropain) subunit\| beta type\| 5 | D29011 | 466.82 | 126.17 | 27.03 |
| 38074_at | adaptor-related protein complex 3\| sigma 1 subunit | U91932 | 458.09 | 127.37 | 27.80 |
| 2093_s_at | X-ray repair complementing defective repair in Chinese hamster cells | J04977 | 456.87 | 123.57 | 27.05 |
| | 5 (double-strand-break rejoining; Ku autoantigen\| 80 kD) | | | | |
| 1707_g_at | v-raf murine sarcoma 3611 viral oncogene homolog 1 | U01337 | 451.30 | 123.34 | 27.33 |
| 38050_at | KIAA0164 gene product | D79986 | 427.55 | 118.37 | 27.69 |
| 33770_at | conserved helix-loop-helix ubiquitous kinase | AF009225 | 389.91 | 105.78 | 27.13 |
| 584_s_at | X-ray repair complementing defective repair in Chinese hamster cells | M30938 | 367.41 | 100.27 | 27.29 |
| | 5 (double-strand-break rejoining; Ku autoantigen\| 80 kD) | | | | |
| 33860_at | KIAA0462 protein | AB007931 | 366.96 | 102.39 | 27.90 |
| 37318_at | eukaryotic translation termination factor 1 | X81625 | 365.75 | 101.16 | 27.66 |
| 34680_s_at | KIAA0107 gene product | D14663 | 362.96 | 100.09 | 27.58 |
| 38713_at | EST | Z99716 | 361.32 | 98.49 | 27.26 |
| 41267_at | KIAA1049 protein | AB028972 | 357.38 | 97.24 | 27.21 |
| 32572_at | ubiquitin specific protease 9\| X chromosome (Drosophila fat facets | X98296 | 332.71 | 90.61 | 27.23 |
| | related) | | | | |
| 34374_g_at | upstream regulatory element binding protein 1 | Z97054 | 321.11 | 88.15 | 27.45 |
| 39711_at | protein kinase C substrate 80K-H | J03075 | 319.60 | 87.78 | 27.47 |
| 35268_at | hypothetical protein DKFZp586F1122 similar to axotrophin | AL050171 | 313.65 | 87.38 | 27.86 |
| 35653_at | G protein pathway suppressor 2 | U28963 | 310.61 | 85.73 | 27.60 |
| 34326_at | coatomer protein complex\| subunit beta | X82103 | 308.28 | 84.76 | 27.49 |
| 641_at | presenilin 1 (Alzheimer disease 3) | L76517 | 306.24 | 85.17 | 27.81 |
| 38993_r_at | EST | W27522 | 291.43 | 79.75 | 27.36 |
| 36971_at | KIAA0257 protein | D87446 | 278.89 | 77.69 | 27.86 |
| 421_at | translocated promoter region (to activated MET oncogene) | X66397 | 258.24 | 71.72 | 27.77 |
| 39108_at | lanosterol synthase (2\|3-oxidosqualene-lanosterol cyclase) | U22526 | 254.13 | 69.15 | 27.21 |
| 35987_g_at | member of MYST family histone acetyl transferases\| homolog of | AL050395 | 238.29 | 65.51 | 27.49 |
| | Drosophila MOF | | | | |
| 41800_s_at | tetratricopeptide repeat domain 2 | U46571 | 234.15 | 65.39 | 27.93 |
| 36463_at | BCL2-associated athanogene 5 | AB020680 | 230.63 | 62.39 | 27.05 |
| 40169_at | cargo selection protein (mannose 6 phosphate receptor binding | AF057140 | 219.28 | 60.40 | 27.54 |
| | protein) | | | | |
| 32594_at | aspartylglucosaminidase\| chaperonin containing TCP1\| subunit 4 | AF026291 | 214.02 | 57.89 | 27.05 |
| | (delta) | | | | |
| 237_s_at | protein phosphatase 2 (formerly 2A)\| catalytic subunit\| alpha isoform | M60483 | 207.99 | 56.46 | 27.15 |
| 39659_at | Ts translation elongation factor\| mitochondrial | L37936 | 195.72 | 53.60 | 27.38 |
| 39083_at | ubiquitin-conjugating enzyme E2D 3 (homologous to yeast UBC4/5) | U39318 | 185.17 | 51.55 | 27.84 |
| 1885_at | excision repair cross-complementing rodent repair deficiency\| | M31899 | 179.82 | 48.99 | 27.24 |
| | complementation group 3 (xeroderma pigmentosum group B | | | | |
| | complementing) | | | | |
| 35166_at | Down syndrome critical region gene 3 | D87343 | 154.33 | 41.98 | 27.20 |
| 31829_r_at | trans-Golgi network protein (46\| 48\| 51 kD isoforms) | AF027515 | 147.16 | 41.05 | 27.90 |
| 41063_g_at | meningioma expressed antigen 5 (hyaluronidase) | AA037278 | 139.74 | 39.09 | 27.97 |
| 38899_s_at | hypothetical protein FLJ20693 | U95822 | 134.53 | 37.33 | 27.75 |
| 40886_at | PRO2047 protein\| eukaryotic translation elongation factor 1 alpha 1- | L41498 | 2553.34 | 716.99 | 28.08 |
| | like 14 | | | | |
| 37449_i_at | guanine nucleotide binding protein (G protein)\| alpha stimulating | X04409 | 2542.93 | 727.52 | 28.61 |
| | activity polypeptide 1 | | | | |
| 32334_f_at | ubiquitin C | AB009010 | 2484.94 | 698.40 | 28.11 |
| 36358_at | ribosomal protein L9 | U09953 | 2319.22 | 664.47 | 28.65 |
| 41178_at | ribosomal protein L11 | X79234 | 2308.70 | 647.00 | 28.02 |
| 33667_at | peptidylprolyl isomerase A (cyclophilin A) | X52851 | 2299.52 | 654.15 | 28.45 |
| 1315_at | omithine decarboxylase antizyme 1 | D78361 | 2286.84 | 658.74 | 28.81 |
| 32435_at | ribosomal protein L19 | X63527 | 2284.19 | 646.33 | 28.30 |
| 32437_at | ribosomal protein S5 | U14970 | 2225.71 | 640.95 | 28.80 |
| 32341_f_at | ribosomal protein L23a | U37230 | 2137.05 | 608.03 | 28.45 |
| 33614_at | ribosomal protein L18a | X80822 | 2104.73 | 609.73 | 28.97 |
| 34608_at | guanine nucleotide binding protein (G protein)\| beta polypeptide 2-like 1 | M24194 | 2100.39 | 601.81 | 28.65 |
| 41741_at | RNA binding motif protein 3 | U28686 | 2093.05 | 597.35 | 28.54 |
| 33659_at | cofilin 1 (non-muscle) | X95404 | 1893.82 | 531.98 | 28.09 |
| 31546_at | ribosomal protein L18 | L11566 | 1779.05 | 510.46 | 28.69 |
| 32432_f_at | ribosomal protein L15 | L25899 | 1729.12 | 501.16 | 28.98 |
| 31584_at | tumor protein\| translationally-controlled 1 | X16064 | 1723.64 | 485.94 | 28.19 |
| 1161_at | heat shock 90 kD protein 1\| beta | J04988 | 1508.47 | 430.13 | 28.51 |
| 1980_s_at | non-metastatic cells 2\| protein (NM23B) expressed in | X58965 | 1466.91 | 416.32 | 28.38 |
| 38708_at | RAN\| member RAS oncogene family | AF054183 | 1453.07 | 410.77 | 28.27 |
| 32340_s_at | nuclease sensitive element binding protein 1 | M85234 | 1379.82 | 391.42 | 28.37 |
| 36668_at | diaphorase (NADH) (cytochrome b-5 reductase) | M28713 | 1361.73 | 389.25 | 28.59 |
| 37003_at | CD63 antigen (melanoma 1 antigen) | X62654 | 1321.50 | 378.24 | 28.62 |
| 38590_r_at | prothymosin\| alpha (gene sequence 28) | M14630 | 1319.89 | 372.46 | 28.22 |
| 1424_s_at | tyrosine 3-monooxygenase/tryptophan 5-monooxygenase activation | D78577 | 1319.87 | 376.56 | 28.53 |
| | protein\| eta polypeptide | | | | |
| 1235_at | tyrosine 3-monooxygenase/tryptophan 5-monooxygenase activation | M86400 | 1317.80 | 376.87 | 28.60 |
| | protein\| zeta polypeptide | | | | |
| 37675_at | solute carrier family 25 (mitochondrial carrier; phosphate carrier)\| | X60036 | 1270.20 | 360.95 | 28.42 |
| | member 3 | | | | |
| 39921_at | cytochrome c oxidase subunit Vb | AI526089 | 1230.78 | 345.45 | 28.07 |
| 32166_at | KIAA1027 protein\|talin | AB028950 | 1196.59 | 342.87 | 28.65 |
| 38485_at | NADH dehydrogenase (ubiquinone) 1\| subcomplex unknown\| 1 (6 kD\| | AA760866 | 1186.58 | 343.63 | 28.96 |
| | KFYI) | | | | |
| 40777_at | catenin (cadherin-associated protein)\| beta 1 (88 kD) | X87838 | 1009.82 | 284.29 | 28.15 |
| 35747_at | stromal cell derived factor receptor 1 | AF035287 | 990.74 | 279.13 | 28.17 |
| 38479_at | acidic protein rich in leucines | Y07969 | 988.91 | 282.66 | 28.58 |
| 1817_at | prefoldin 5 | D89667 | 963.78 | 274.72 | 28.50 |
| 40875_s_at | small nuclear ribonucleoprotein 70 kD polypeptide (RNP antigen) | X06815 | 929.39 | 260.44 | 28.02 |
| 40134_at | ATP synthase\| H+ transporting\| mitochondrial F0 complex\| subunit f\| | AF047436 | 899.04 | 253.99 | 28.25 |
| | isoform 2 | | | | |
| 1199_at | eukaryotic translation initiation factor 4A\| isoform 1 | D13748 | 892.85 | 253.08 | 28.35 |
| 32576_at | eukaryotic translation initiation factor 3\| subunit 5 (epsilon\| 47 kD) | U94855 | 843.99 | 238.11 | 28.21 |
| 40898_at | sequestosome 1 | U46751 | 831.41 | 238.14 | 28.64 |
| 35767_at | ganglioside expression factor 2 | AI565760 | 783.62 | 223.09 | 28.47 |
| 922_at | protein phosphatase 2 (formerly 2A)\| regulatory subunit A (PR 65)\| | J02902 | 780.98 | 222.67 | 28.51 |
| | alpha isoform | | | | |
| 35770_at | ATPase\| H+ transporting\| lysosomal (vacuolar proton pump)\| subunit 1 | D16469 | 774.14 | 217.42 | 28.09 |
| 39867_at | Tu translation elongation factor\| mitochondrial | S75463 | 737.22 | 210.58 | 28.56 |
| 38779_r_at | hepatoma-derived growth factor (high-mobility group protein 1-like) | D16431 | 733.82 | 207.97 | 28.34 |
| 38480_s_at | ubiquitin-conjugating enzyme E2I (homologous to yeast UBC9) | U66867 | 725.16 | 207.77 | 28.65 |
| 40874_at | endothelial differentiation-related factor 1 | AJ005259 | 686.91 | 197.37 | 28.73 |
| 32408_s_at | EST | AL022101 | 686.55 | 193.43 | 28.17 |
| 36950_at | gp25L2 protein\|sulfotransferase family\| cytosolic\| 1C\| member 2 | X90872 | 652.46 | 184.68 | 28.30 |
| 41268_g_at | KIAA1049 protein | AB028972 | 645.47 | 187.01 | 28.97 |
| 31932_f_at | basic transcription factor 3 | M90357 | 635.47 | 178.99 | 28.17 |
| 1310_at | proteasome (prosome\| macropain) subunit\| beta type\| 2 | D26599 | 632.60 | 179.78 | 28.42 |
| 33443_at | EST | Z99129 | 583.58 | 167.76 | 28.75 |
| 38814_at | ATPase\| H+ transporting\| lysosomal (vacuolar proton pump)\| member J | AF038954 | 565.80 | 163.29 | 28.86 |
| 34305_at | poly(rC)-binding protein 1 | Z29505 | 560.63 | 158.60 | 28.29 |
| 34338_at | cytoskeleton-associated protein 1 | D49738 | 557.85 | 160.89 | 28.84 |
| 37569_at | programmed cell death 6 | AF035606 | 557.03 | 160.99 | 28.90 |
| 38778_at | KIAA1046 protein | AB028969 | 556.85 | 158.44 | 28.45 |
| 1728_at | murine leukemia viral (bmi-1) oncogene homolog | L13689 | 541.21 | 156.14 | 28.85 |
| 32803_at | cornichon-like | AF104398 | 536.76 | 153.44 | 28.59 |
| 37729_at | exportin 1 (CRM1\| yeast\| homolog) | Y08614 | 523.31 | 151.12 | 28.88 |
| 585_at | X-ray repair complementing defective repair in Chinese hamster cells | M30938 | 522.85 | 150.23 | 28.73 |
| | 5 (double-strand-break rejoining; Ku autoantigen\| 80 kD) | | | | |
| 882_at | colony stimulating factor 1 (macrophage) | M37435 | 511.72 | 143.88 | 28.12 |
| 39370_at | Microtubule-associated proteins 1A and 1B\| light chain 3 | W28807 | 507.65 | 143.26 | 28.22 |
| 41212_r_at | Williams-Beuren syndrome chromosome region 1 | D26068 | 488.87 | 137.63 | 28.15 |
| 33877_s_at | KIAA1067 protein | AB028990 | 486.65 | 136.63 | 28.07 |
| 1446_at | proteasome (prosome\| macropain) subunit\| alpha type\| 2 | D00760 | 472.98 | 133.17 | 28.16 |
| 41197_at | RAD23 (S. cerevisiae) homolog A | D21235 | 459.28 | 132.47 | 28.84 |
| 37300_at | dynein\| cytoplasmic\| heavy polypeptide 1 | AB002323 | 446.27 | 126.74 | 28.40 |
| 1030_s_at | topoisomerase (DNA)\| | U07806 | 444.60 | 128.72 | 28.95 |
| 38282_at | a disintegrin and metalloproteinase domain 15 (metargidin) | U41767 | 424.89 | 120.16 | 28.28 |
| 32569_at | platelet-activating factor acetylhydrolase\| isoform lb\| alpha subunit | L13385 | 424.73 | 119.93 | 28.24 |
| | (45 kD) | | | | |
| 36845_at | KIAA0136 protein | D50926 | 401.91 | 115.00 | 28.61 |
| 32853_at | translocase of outer mitochondrial membrane 70 (yeast) homolog A | AB018262 | 388.05 | 111.34 | 28.69 |
| 37860_at | DKFZP564F1422 protein | AL049942 | 379.05 | 107.48 | 28.35 |
| 32209_at | Mouse Mammary Turmor Virus Receptor homolog | AF052151 | 374.27 | 105.88 | 28.29 |
| 39029_at | matemal G10 transcript | U11861 | 368.78 | 106.14 | 28.78 |
| 1313_at | proteasome (prosome\| macropain) subunit\| beta type\| 7 | D38048 | 359.96 | 101.82 | 28.29 |
| 1398_g_at | mitogen-activated protein kinase kinase kinase 11 | L32976 | 351.84 | 100.27 | 28.50 |
| 35263_at | glutathione S-transferase M1 | N73769 | 350.90 | 100.48 | 28.64 |
| 37931_at | centromere protein B (80 kD) | X05299 | 350.36 | 101.52 | 28.98 |
| 35836_at | nuclear distribution gene C (A. nidulans) homolog | AB019408 | 346.50 | 98.88 | 28.54 |
| 33424_at | ribophorin I | Y00281 | 345.17 | 99.65 | 28.87 |
| 37670_at | annexin A7 | J04543 | 339.52 | 96.40 | 28.39 |
| 869_at | general transcription factor IIA\| 2 (12 kD subunit) | U14193 | 325.31 | 93.67 | 28.80 |
| 32117_at | apoptosis antagonizing transcription factor | U51698 | 316.94 | 90.54 | 28.57 |
| 41316_s_at | scaffold attachment factor B | U72355 | 287.71 | 83.12 | 28.89 |
| 38705_at | ubiquitin-conjugating enzyme E2D 2 (homologous to yeast UBC4/5) | AI310002 | 284.21 | 81.17 | 28.56 |
| 40615_at | hypothetical protein FLJ21439 | AA780049 | 265.07 | 74.85 | 28.24 |
| 38475_at | dynactin 2 (p50) | U50733 | 260.78 | 74.16 | 28.44 |
| 504_at | ubiquitin-conjugating enzyme E2D 3 (homologous to yeast UBC4/5) | U39318 | 259.05 | 73.73 | 28.46 |
| 34692_r_at | actin related protein 2/3 complex\| subunit 4 (20 kD) | AF006087 | 254.79 | 71.76 | 28.16 |
| 37703_at | Rab geranylgeranyltransferase\| beta subunit | Y08201 | 244.42 | 69.34 | 28.37 |
| 41604_at | EST | U79297 | 241.37 | 68.58 | 28.41 |
| 32205_at | protein kinase\| interferon-inducible double stranded RNA dependent | AF072860 | 241.35 | 68.80 | 28.50 |
| | activator | | | | |
| 37911_at | syntaxin 4A (placental) | U07158 | 238.94 | 68.91 | 28.84 |
| 1074_at | RAB1\| member RAS oncogene family | M28209 | 236.35 | 66.47 | 28.12 |
| 41097_at | telomeric repeat binding factor 2 | AF002999 | 213.33 | 61.81 | 28.98 |
| 39368_at | eukaryotic translation initiation factor 2\| subunit 2 (bete\| 38 kD) | AL031668 | 209.73 | 59.84 | 28.53 |
| 39141_at | ATP-binding cassette\| sub-family F (GCN20)\| member 1 | AF027302 | 192.53 | 54.74 | 28.43 |
| 41699_f_at | bromodomain-containing 1 | AL080149 | 190.86 | 54.73 | 28.68 |
| 1453_at | MAD (mothers against decapentaplegic\| Drosophila) homolog 2 | U68018 | 189.69 | 53.39 | 28.15 |
| 34705_at | similar to yeast BET3 (S. cerevisiae) | AJ224335 | 178.32 | 50.74 | 28.46 |
| 39342_at | methionine-tRNA synthetase | X94754 | 174.57 | 50.36 | 28.85 |
| 35368_at | zinc finger protein 207 | AF046001 | 153.79 | 44.08 | 28.67 |
| 33841_at | hypothetical protein FLJ11560 | R48209 | 140.73 | 39.88 | 28.34 |
| 39403_at | KIAA0678 protein | AB014578 | 127.16 | 35.83 | 28.17 |
| 193_at | TATA box binding protein (TBP)-associated factor\| RNA polymerase | U21858 | 124.45 | 35.43 | 28.47 |
| | II\| G\| 32 kD | | | | |
| 40537_at | KIAA0741 gene product | AB018284 | 119.09 | 33.47 | 28.10 |
| 35286_r_at | putative nucleic acid binding protein RY-1 | X76302 | 104.69 | 30.35 | 28.99 |
| 35119_at | ribosomal protein L13a | X56932 | 2374.45 | 697.62 | 29.38 |
| 31568_at | ribosomal protein S10 | U14972 | 2240.21 | 649.67 | 29.00 |
| 32330_at | ribosomal protein S11 | X06617 | 2087.27 | 608.76 | 29.17 |
| 34592_at | ribosomal protein S17 | M13932 | 2041.57 | 595.03 | 29.15 |
| 32436_at | ribosomal protein L27a | U14968 | 1911.48 | 560.72 | 29.33 |
| 33656_at | ribosomal protein L37 | D23661 | 1861.14 | 549.25 | 29.51 |
| 33668_at | ribosomal protein L12 | AF037643 | 1553.32 | 461.37 | 29.70 |
| 39798_at | ribosomal protein S28 | R87876 | 1506.80 | 443.11 | 29.41 |
| 1718_at | actin related protein 2/3 complex\| subunit 2 (34 kD) | U50523 | 1429.15 | 418.75 | 29.30 |
| 37307_at | guanine nucleotide binding protein (G protein)\| alpha inhibiting activity | X04828 | 1284.55 | 379.25 | 29.52 |
| | polypeptide 2 | | | | |
| 39027_at | cytochrome c oxidase subunit IV | AF017115 | 1271.56 | 372.18 | 29.27 |
| 955_at | EST | HG1862-HT1897 | 977.60 | 283.88 | 29.04 |
| 41220_at | MLL septin-like fusion | AB023208 | 960.99 | 279.78 | 29.11 |
| 38075_at | synaptophysin-like protein | X68194 | 896.44 | 267.11 | 29.80 |
| 39033_at | chromosome 1 open reading frame 8 | Z78368 | 856.61 | 253.99 | 29.65 |
| 32324_at | tyrosine 3-monooxygenase/tryptophan 5-monooxygenase activation | X57346 | 818.90 | 240.26 | 29.34 |
| | protein\| beta polypeptide | | | | |
| 40593_at | polypyrimidine tract binding protein (heterogeneous nuclear | X66975 | 803.31 | 239.11 | 29.77 |
| | ribonucleoprotein I) | | | | |
| 41235_at | activating transcription factor 4 (tax-responsive enhancer element | AL022312 | 793.91 | 233.65 | 29.43 |
| | B67). | | | | |
| 32774_at | NADH dehydrogenase (ubiquinone) 1 beta subcomplex\| 8 (19 kD\| | AI541050 | 731.80 | 215.75 | 29.48 |
| | ASHI) | | | | |
| 31492_at | muscle specific gene | AB019392 | 684.80 | 202.42 | 29.56 |
| 41834_g_at | jumping translocation breakpoint | AB016492 | 664.62 | 198.64 | 29.89 |
| 32335_r_at | ubiquitin C | AB009010 | 626.97 | 185.42 | 29.57 |
| 41233_at | MRJ gene for a member of the DNAJ protein family | AB014888 | 626.57 | 185.50 | 29.61 |
| 40783_s_at | phosphatidylinositol 4-kinase\| catalytic\| alpha polypeptide | L36151 | 623.93 | 184.30 | 29.54 |
| 34796_at | translocating chain-associating membrane protein | X63679 | 610.94 | 177.90 | 29.12 |
| 41202_s_at | conserved gene amplified in osteosarcoma | AF000152 | 603.25 | 178.02 | 29.51 |
| 40867_at | protein phosphatase 2 (formerly 2A)\| regulatory subunit A (PR 65)\| | J02902 | 591.17 | 173.56 | 29.36 |
| | alpha isoform | | | | |
| 2050_s_at | ras-related C3 botulinum toxin substrate 1 (rho family\| small GTP | M29870 | 583.02 | 169.72 | 29.11 |
| | binding protein Rac1) | | | | |
| 39363_at | putative breast adenocarcinoma marker (32 kD) | AF042384 | 539.48 | 157.47 | 29.19 |
| 34791_at | t-complex 1 | X52882 | 535.37 | 159.39 | 29.77 |
| 38375_at | esterase D/formylglutathione hydrolase | AF112219 | 512.06 | 149.48 | 29.19 |
| 40467_at | succinate dehydrogenase complex\| subunit D\| integral membrane | AB006202 | 511.52 | 149.42 | 29.21 |
| | protein | | | | |
| 32478_f_at | EST | AL031133 | 504.81 | 146.84 | 29.09 |
| 38690_at | chromosome 3 open reading frame 4 | AL080097 | 503.41 | 146.77 | 29.15 |
| 40106_at | E1B-55 kDa-associated protein 5 | AJ007509 | 497.05 | 146.61 | 29.50 |
| 37387_r_at | KDEL (Lys-Asp-Glu-Leu) endoplasmic reticulum protein retention | X55885 | 469.97 | 139.61 | 29.71 |
| | receptor 1 | | | | |
| 40108_at | KIAA0005 gene product | D13630 | 429.34 | 127.15 | 29.61 |
| 32235_at | KIAA0544 protein | AB011116 | 421.84 | 124.11 | 29.42 |
| 40546_s_at | NADH dehydrogenase (ubiquinone) 1 alpha subcomplex\| 2 (8 kD\| B8) | AF047185 | 415.67 | 122.99 | 29.59 |
| 41594_at | Janus kinase 1 (a protein tyrosine kinase) | M64174 | 415.30 | 122.67 | 29.54 |
| 35983_at | EST | AC004528 | 409.89 | 119.89 | 29.25 |
| 36637_at | annexin A11 | L19605 | 404.86 | 118.94 | 29.38 |
| 40225_at | cyclin G associated kinase | D88435 | 385.87 | 114.69 | 29.72 |
| 38744_at | Deleted in split-hand/split-foot 1 region | N95406 | 373.10 | 109.49 | 29.35 |
| 32221_at | PTD017 protein | AL050361 | 371.70 | 108.89 | 29.30 |
| 1314_at | proteasome (prosome\| macropain) 26S subunit\| non-ATPase\| 1 | D44466 | 368.61 | 109.63 | 29.74 |
| 36208_at | bromodomain-containing 2 | D42040 | 360.93 | 105.61 | 29.26 |
| 140_s_at | splicing factor\| arginine/serine-rich (transformer 2 Drosophila | U68063 | 360.19 | 106.20 | 29.48 |
| | homolog) 10 | | | | |
| 40979_at | chromosome 14 open reading frame 3 | AJ243310 | 355.27 | 106.33 | 29.93 |
| 40610_at | M-phase phosphoprotein homolog | AI743507 | 344.58 | 101.36 | 29.42 |
| 38703_at | aspartyl aminopeptidase | AF005050 | 339.21 | 100.19 | 29.54 |
| 38399_at | small nuclear ribonucleoprotein polypeptide B″ | AL034428 | 337.91 | 101.05 | 29.90 |
| 38982_at | TRF2-interacting telomeric RAP1 protein | W28865 | 332.80 | 96.76 | 29.07 |
| 1512_at | dual-specificity tyrosine-(Y)-phosphorylation regulated kinase 1A | D86550 | 330.61 | 97.02 | 29.35 |
| 38648_at | trinucleotide repeat containing 1 | U80760 | 322.64 | 96.15 | 29.80 |
| 37731_at | epidermal growth factor receptor pathway substrate 15 | Z29064 | 319.59 | 95.65 | 29.93 |
| 38472_at | KIAA0143 protein | D63477 | 318.06 | 94.20 | 29.62 |
| 38613_at | putative cyclin G1 interacting protein | U61837 | 315.51 | 93.79 | 29.73 |
| 39686_g_at | like mouse brain protein E46 | AL050282 | 314.96 | 91.65 | 29.10 |
| 891_at | YY1 transcription factor | M77698 | 313.42 | 93.44 | 29.81 |
| 38689_at | hypothetical protein | AL021937 | 290.41 | 86.38 | 29.74 |
| 37517_at | KIAA1039 protein | AB028962 | 287.85 | 85.63 | 29.75 |
| 34861_at | golgi autoantigen\| golgin subfamily a\| 3 | D63997 | 281.99 | 82.38 | 29.21 |
| 39823_at | H326 | U06631 | 280.35 | 81.71 | 29.15 |
| 37581_at | protein phosphatase 6\| catalytic subunit | X92972 | 270.45 | 78.94 | 29.19 |
| 38977_at | tyrosyl-tRNA synthetase | U89436 | 262.14 | 77.20 | 29.45 |
| 41763_g_at | TIA1 cytotoxic granule-associated RNA-binding protein-like 1 | D64015 | 261.11 | 78.14 | 29.93 |
| 37690_at | ilvB (bacterial acetolactate synthase)-like | U61263 | 259.17 | 76.96 | 29.70 |
| 34323_at | thyroid receptor interacting protein 15 | AF084260 | 259.01 | 75.79 | 29.26 |
| 33706_at | squamous cell carcinoma antigen recognised by T cells | AB006198 | 255.89 | 74.35 | 29.06 |
| 40132_g_at | follistatin-like 1 | D89937 | 253.42 | 74.99 | 29.59 |
| 34753_at | synaptobrevin-like 1 | X92396 | 252.85 | 75.48 | 29.85 |
| 39601_at | Ras association (RalGDS/AF-6) domain family 1 | AF061836 | 251.76 | 74.05 | 29.41 |
| 34385_at | succinate dehydrogenase complex\| subunit C\| integral membrane | U57877 | 244.41 | 73.09 | 29.91 |
| | protein\| 15 kD | | | | |
| 33301_g_at | cell division cycle 2-like 1 (PITSLRE proteins) | AL031282 | 243.26 | 71.81 | 29.52 |
| 35301_at | EST | AL049941 | 236.41 | 69.61 | 29.44 |
| 36535_at | microfibrillar-associated protein 1 | U04209 | 211.33 | 61.55 | 29.13 |
| 34733_at | splicing factor 3a\| subunit 1\| 120 kD | X85237 | 210.62 | 61.13 | 29.02 |
| 33268_at | SMC (mouse) homolog\| X chromosome | L25270 | 201.57 | 59.58 | 29.56 |
| 39746_at | polymerase (RNA) ∥ (DNA directed) polypeptide B (140 kD) | X63563 | 197.91 | 58.18 | 29.39 |
| 39380_at | KIAA0697 protein | AB014597 | 195.04 | 58.16 | 29.82 |
| 40605_at | sorting nexin 4 | AA524345 | 189.00 | 55.74 | 29.49 |
| 949_s_at | proteasome (prosome\| macropain) 26S subunit\| ATPase\| 6 | D78275 | 186.24 | 55.24 | 29.66 |
| 40623_at | ubiquitin protein ligase | AI749193 | 182.34 | 53.45 | 29.31 |
| 37907_at | coagulation factor VIII-associated (intronic transcript) | M34677 | 181.01 | 53.35 | 29.47 |
| 229_at | CCAAT-box-binding transcription factor | M37197 | 112.31 | 33.26 | 29.61 |
| 37385_at | Clk-associating RS-cyclophilin | U40763 | 86.32 | 25.20 | 29.19 |

TABLE 2

| Fragment | | Mean | | % CV | % CV |
|---|---|---|---|---|---|
| No. | Gene Name | Ave. Diff. | St_dev | low PMT | high PMT |
| 39415_at | H. sapiens tunp mRNA for transformation upregulated nuclear | 1026.88809 | 170.565203 | 17% | 23% |
| | protein. | | | | |
| 41194_at | PT1.3_04_C04.r tumor1 Homo sapiens cDNA 5′, mRNA sequence. | 1644.61573 | 279.769383 | 17% | 21% |
| 33987_at | Human ADP-ribosylation factor 1 (ARF1) mRNA, complete cds. | 1053.60427 | 184.57965 | 18% | 24% |
| 32575_at | Human nucleosome assembly protein 2 mRNA, complete cds. | 477.012 | 84.240391 | 18% | 22% |
| 41785_at | Human p97 mRNA, complete cds. | 2146.16736 | 412.628482 | 19% | 22% |
| 39346_at | Human p62 mRNA, complete cds. | 546.721182 | 107.321822 | 20% | 26% |
| 31952_at | H. sapiens mRNA for ribosomal protein L6. | 3206.74227 | 640.283429 | 20% | 26% |
| | ns20e08.s1 NCl_CGAP_GCB1 Homo sapiens cDNA clone | | | | |
| 38084_at | IMAGE:1184198 3′, mRNA sequence. | 1118.78036 | 232.129292 | 21% | 28% |
| 35292_at | H. sapiens BAT1 mRNA for nuclear RNA helicase (DEAD family). | 795.261727 | 167.254169 | 21% | 26% |
| 39739_at | Homo sapiens alpha NAC mRNA, complete cds. | 2243.61464 | 472.024126 | 21% | 27% |
| 33619_at | Human ribosomal protein S13 (RPS13) mRNA, complete cds. | 3007.25591 | 638.766828 | 21% | 28% |
| 38817_at | Homo sapiens sperm acrosomal protein mRNA, complete cds. | 421.342818 | 90.0824915 | 21% | 30% |
| 37321_at | Human tetratricopeptide repeat protein (tpr1) mRNA, complete cds. | 354.162182 | 76.0661789 | 21% | 26% |
| 39027_at | Homo sapiens cytochrome c oxidase subunit IV precursor (COX4) | 1335.39746 | 286.945221 | 21% | 29% |
| | gene, nuclear gene encoding mitochondrial protein, complete cds. | | | | |
| 40593_at | H. sapiens mRNA for heterogeneous nuclear ribonucleoprotein. | 1078.26327 | 231.966596 | 22% | 30% |
| 35302_at | Homo sapiens mRNA for TAP/NXF1 protein (nxf1 gene). | 334.106818 | 71.8899126 | 22% | 26% |
| 34608_at | Human MHC protein homologous to chicken B complex protein | 5005.15818 | 1077.96802 | 22% | 29% |
| | mRNA, complete cds. | | | | |
| 34570_at | ubiquitin carboxyl extension protein [human, mRNA, 540 nt]. | 2283.91418 | 493.661115 | 22% | 28% |
| 1653_at | Human v-fos transformation effector protein (Fte-1), mRNA complete | 3990.42273 | 869.139812 | 22% | 28% |
| | cds | | | | |
| 33656_at | Human mRNA for ribosomal protein L37, complete cds. | 3596.03609 | 785.088807 | 22% | 30% |
| | DKFZp761M078_s1 761 (synonym: hamy2) Homo sapiens cDNA | | | | |
| 33826_at | clone DKFZp761M078 3′, mRNA sequence. | 294.780091 | 64.3590978 | 22% | 22% |
| 36166_at | Homo sapiens SPF31 (SPF31) mRNA, complete cds. | 226.214546 | 49.7114097 | 22% | 25% |
| 41224_at | Homo sapiens mRNA for KIAA0788 protein, partial cds. | 564.298 | 125.081423 | 22% | 25% |
| 35055_at | H. sapiens BTF3b mRNA. | 2535.722 | 562.331297 | 22% | 24% |
| 31708_at | Homo sapiens ribosomal protein L30 mRNA, complete cds. | 3124.15355 | 692.861641 | 22% | 27% |
| 36972_at | H. sapiens mRNA for transmembrane protein rnp24. | 866.941455 | 192.916911 | 22% | 23% |
| 32337_at | Human ribosomal protein L21 mRNA, complete cds. | 2134.87055 | 475.979644 | 22% | 27% |
| 254_at | Human H3.3 histone class C mRNA, complete cds. | 1413.40964 | 317.087392 | 22% | 24% |
| 33660_at | Human ribosomal protein L5 mRNA, complete cds. | 2931.42864 | 660.516812 | 23% | 26% |
| 32220_at | Human mRNA for HMG-1, complete cds. | 746.693909 | 168.262743 | 23% | 25% |
| 34646_at | H. sapiens gene for ribosomal protein S7. | 2500.63473 | 568.252652 | 23% | 25% |
| 40211_at | Human gene for heterogeneous nuclear ribonucleoprotein (hnRNP) | 1574.12555 | 360.039065 | 23% | 31% |
| | core protein A1. | | | | |
| 37581_at | H. sapiens mRNA for protein phosphatase 6. | 232.166273 | 53.1072301 | 23% | 29% |
| 38689_at | Cluster Incl AL021937: dJ149A16.6 (novel protein, human ortholog of | 285.348909 | 65.3091428 | 23% | 30% |
| | worm F16A11.2 and bacterial and archea-bacterial predicted | | | | |
| | proteins)/cds = (84,1601)/gb = AL021937/gi = 4165210/ug = Hs. 10729/ | | | | |
| | len = 2014 | | | | |
| 38437_at | H. sapiens MLN51 mRNA. | 418.190273 | 95.7712211 | 23% | 31% |
| 505_at | Human CDC37 homolog mRNA, complete cds. | 670.117455 | 154.995532 | 23% | 22% |
| 35125_at | H. sapiens gene for ribosomal protein S6. | 3232.79191 | 747.844258 | 23% | 28% |
| 1315_at | Human mRNA for ornithine decarboxylase antizyme, ORF 1 and | 4306.74709 | 998.152867 | 23% | 29% |
| | ORF2. | | | | |
| 38672_at | Homo sapiens fb19 mRNA. | 158.562273 | 36.7548606 | 23% | 22% |
| | qp51f08.x1 NCI_CGAP_Co8 Homo sapiens cDNA clone | | | | |
| 37389_at | IMAGE: 1926567 3′ similar to TR: O00193 O00193 SMALL ACIDIC | 414.294546 | 96.1843904 | 23% | 25% |
| | PROTEIN.;, mRNA sequence. | | | | |
| 34861_at | Homo sapiens mRNA for GCP170, complete cds. | 211.268818 | 49.1374269 | 23% | 29% |
| 35119_at | H. sapiens mRNA for 23 kD highly basic protein. | 5442.70309 | 1279.01272 | 23% | 29% |
| 31385_at | Human ribosomal protein L28 mRNA, complete cds. | 6173.96791 | 1453.22923 | 24% | 31% |
| 32437_at | Human ribosomal protein S5 mRNA, complete cds. | 4776.60091 | 1129.61556 | 24% | 29% |
| 1009_at | Homo sapiens protein kinase C inhibitor (PKCI-1) mRNA, complete | 1840.12073 | 435.694395 | 24% | 27% |
| | cds. | | | | |
| 33677_at | Homo sapiens ribosomal protein L30 mRNA, complete cds. | 3565.723 | 844.726812 | 24% | 27% |
| 40637_at | Human hsc70 gene for 71 kd heat shock cognate protein. | 1347.46027 | 319.242248 | 24% | 26% |
| 32436_at | Human ribosomal protein L27a mRNA, complete cds. | 3859.96082 | 916.763496 | 24% | 29% |
| 32436_at | zk72a10.s1 Soares_pregnant_uterus_NbHPU Homo sapiens cDNA | 4142.93273 | 984.53904 | 24% | 27% |
| | clone IMAGE: 488346 3′ similar to gb: L19527 60S RIBOSOMAL | | | | |
| | PROTEIN L27 (HUMAN);, mRNA sequence. | | | | |
| 38061_at | pec1.2-3.F11.r ecnorm Homo sapiens cDNA 5′, mRNA sequence. | 4709.65727 | 1120.18549 | 24% | 30% |
| 32553_at | Human zinc finger protein (MAZ) mRNA. | 1324.87791 | 317.050776 | 24% | 30% |
| 32330_at | Human mRNA for ribosomal protein S11. | 4781.80118 | 1145.31708 | 24% | 29% |
| 38483_at | Homo sapiens mRNA for hypothetical protein. | 741.475091 | 177.620755 | 24% | 22% |
| 33668_at | Homo sapiens 60S ribosomal protein L12 (RPL12) pseudogene, | 3666.22155 | 879.972991 | 24% | 30% |
| | partial sequence. | | | | |
| 36003_at | Homo sapiens mRNA for poly(A)-specific ribonuclease. | 152.459182 | 36.7579511 | 24% | 24% |
| 39047_at | Homo sapiens mRNA for squamous cell carcinoma antigen SART-3, | 250.677909 | 60.6222473 | 24% | 24% |
| | complete cds. | | | | |
| | zu48g06.r1 Soares ovary tumor NbHOT Homo sapiens cDNA clone | | | | |
| 39811_at | IMAGE: 741274 5′, mRNA sequence. | 462.080364 | 111.804544 | 24% | 21% |
| 33614_at | H. sapiens mRNA for ORF. | 4438.22655 | 1076.78347 | 24% | 29% |
| 38046_at | Homo sapiens mRNA for Prer protein. | 189.351636 | 45.9816717 | 24% | 30% |
| 36186_at | Human (clone E5.1) RNA-binding protein mRNA, complete cds. | 1000.01436 | 242.987057 | 24% | 25% |
| 38527_at | Human 54 kDa protein mRNA, complete cds. | 1172.11364 | 285.380767 | 24% | 27% |
| 40426_at | H. sapiens mRNA for BCL7B protein. | 264.450818 | 64.4874354 | 24% | 25% |
| 40125_at | Homo sapiens integral membrane protein, calnexin, (IP90) mRNA, | 1280.86518 | 312.743724 | 24% | 24% |
| | complete cds. | | | | |
| 37675_at | H. sapiens mRNA for mitochondrial phosphate carrier protein. | 1634.062 | 399.04644 | 24% | 28% |
| 31546_at | Homo sapiens ribosomal protein L18 (RPL18) mRNA, complete cds. | 3255.11446 | 796.143304 | 24% | 29% |
| 40469_at | Homo sapiens mRNA for KIAA0572 protein, partial cds. | 228.441546 | 56.0920217 | 25% | 25% |
| 34647_at | Human mRNA for p68 protein. | 662.582455 | 162.814744 | 25% | 30% |
| 32315_at | Human ribosomal protein S24 mRNA. | 3646.17255 | 899.123351 | 25% | 27% |
| 32241_at | Homo sapiens mRNA; cDNA DKFZp564O1716 (from clone | 551.880091 | 136.161164 | 25% | 25% |
| | DKFZp564O1716); complete cds. | | | | |
| 32435_at | H. sapiens mRNA for ribosomal protein L19. | 4612.49427 | 1138.57159 | 25% | 28% |
| 33659_at | H. sapiens mRNA for non-muscle type cofilin. | 4095.13346 | 1011.2557 | 25% | 28% |
| 37309_at | Homo sapiens RHOA proto-oncogene multi-drug-resistance protein | 2244.47327 | 554.267545 | 25% | 23% |
| | mRNA, 3′ end. | | | | |
| 32039_at | Homo sapiens beta-3A-adaptin subunit of the AP-3 complex mRNA, | 202.007546 | 49.9878655 | 25% | 26% |
| | complete cds. | | | | |
| 31573_at | Human ribosomal protein S25 mRNA, complete cds. | 3115.30918 | 775.8861 | 25% | 26% |
| 38974_at | Homo sapiens RNA-binding protein regulatory subunit mRNA, | 1349.68091 | 336.794841 | 25% | 22% |
| | complete cds. | | | | |
| 39727_at | Homo sapiens protein tyrosine phosphatase PIR1 mRNA, complete | 187.273909 | 46.8452354 | 25% | 31% |
| | cds. | | | | |
| 36333_at | H. sapiens mRNA for ribosomal protein L7. | 3379.63209 | 849.347293 | 25% | 26% |
| | qa49c09.x1 Soares_NhHMPu_S1 Homo sapiens cDNA clone | | | | |
| 33861_at | IMAGE: 1690096 3′, mRNA sequence. | 152.208909 | 38.2848254 | 25% | 26% |
| 32841_at | Human nucleic acid binding protein gene, complete cds. | 226.290818 | 56.9256749 | 25% | 31% |
| 41197_at | Human mRNA for HHR23A protein, complete cds. | 478.684091 | 120.69157 | 25% | 29% |
| 38654_at | H. sapiens U21.1 mRNA. | 229.842091 | 57.9601077 | 25% | 30% |
| 41833_at | Homo sapiens hJTB gene, complete cds. | 1343.33582 | 338.936146 | 25% | 22% |
| 36786_at | Cluster Incl AL022721: dJ109F14.2 (60S Ribosomal Protein RPL10A)/ | 2589.09446 | 654.928367 | 25% | 31% |
| | cds = (15,668)/gb = AL022721/gi = 3367610/ug = Hs.76067/len = 703 | | | | |
| 39360_at | Homo sapiens sorting nexin 3 (SNX3) mRNA, complete cds. | 678.488727 | 171.970376 | 25% | 22% |
| 33984_at | Human 90-kDa heat-shock protein gene, cDNA, complete cds. | 2899.13164 | 734.823069 | 25% | 27% |
| 32594_at | Homo sapiens chaperonin containing t-complex polypeptide 1, delta | 229.645182 | 58.2445319 | 25% | 27% |
| | subunit (Cctd) mRNA, complete cds. | | | | |
| 1885_at | Human DNA repair helicase (ERCC3) mRNA, complete cds. | 154.336546 | 39.2181176 | 25% | 27% |
| 39778_at | Human N-acetylglucosaminyltransferase\| (GlcNAc-TI) mRNA, | 256.824727 | 65.2740889 | 25% | 23% |
| | complete cds. | | | | |
| 35298_at | Homo sapiens translation initiation factor elF3 p66 subunit mRNA, | 1044.27864 | 265.763545 | 25% | 28% |
| | complete cds. | | | | |
| 41178_at | H. sapiens mRNA for ribosomal protein L11. | 4617.69046 | 1176.58903 | 25% | 28% |
| 34864_at | Homo sapiens clone 24448 unknown mRNA, partial cds. | 400.608273 | 102.132318 | 25% | 22% |
| | as86g01.x1 Barstead colon HPLRB7 Homo sapiens cDNA clone | | | | |
| 34381_at | IMAGE: 2335632 3′ similar to gb: X16560 CYTOCHROME C | 1715.23391 | 437.988735 | 26% | 27% |
| | OXIDASE POLYPEPTIDE VIIC PRECURSOR (HUMAN);, mRNA | | | | |
| | sequence. | | | | |
| 35835_at | Homo sapiens mRNA, expressed in fibroblasts of periodontal | 536.913 | 137.112927 | 26% | 22% |
| | ligament, complete cds, clone: PDL-108. | | | | |
| 36358_at | Human ribosomal protein L9 mRNA, complete cds. | 3983.14173 | 1018.47768 | 26% | 29% |
| 31509_at | H. sapiens BBC1 mRNA. | 3235.68836 | 828.95099 | 26% | 27% |
| 38542_at | Homo sapiens nucleophosmin phosphoprotein (NPM) gene, 3′ | 3789.96627 | 971.028442 | 26% | 25% |
| | flankinq sequence. | | | | |
| 38708_at | Homo sapiens GTP binding protein mRNA, complete cds. | 3069.247 | 787.553228 | 26% | 28% |
| 38016_at | Homo sapiens hnRNP-C like protein mRNA, complete cds. | 470.094 | 121.294239 | 26% | 24% |
| 34891_at | PEC1.2_15_H01.r ecnorm Homo sapiens cDNA 5′, mRNA | 1695.08746 | 437.605159 | 26% | 26% |
| | sequence. | | | | |
| 41741_at | Human putative RNA binding protein RNPL mRNA, complete cds. | 2346.64009 | 605.819094 | 26% | 29% |
| 33666_at | Human nuclear ribonucleoprotein particle (hnRNP) C protein mRNA, | 857.862 | 221.645044 | 26% | 25% |
| | complete cds. | | | | |
| 33667_at | Human cyclophilin gene for cyclophilin (EC 5.2.1.8). | 5131.12591 | 1326.01063 | 26% | 28% |
| 39141_at | Homo sapiens TNF-alpha stimulated ABC protein (ABC50) mRNA, | 213.669727 | 55.328673 | 26% | 28% |
| | complete cds. | | | | |
| 31583_at | H. sapiens rpS8 gene for ribosomal protein S8. | 4181.05982 | 1083.52675 | 26% | 30% |
| 32440_at | Human L23 mRNA for putative ribosomal protein. | 3094.835 | 802.951872 | 26% | 28% |
| 36137_at | H. sapiens mRNA for 218 kD Mi-2 protein. | 480.124818 | 124.737464 | 26% | 25% |
| | DKFZp434A0418_s1 434 (synonym: htes3) Homo sapiens cDNA | | | | |
| 41295_at | clone DKFZp434A0418 3′, mRNA sequence. | 1030.45891 | 267.908852 | 26% | 23% |
| 39336_at | Human ADP-ribosylation factor 3 mRNA, complete cds. | 670.617182 | 174.637432 | 26% | 26% |
| 632_at | Homo sapiens glycogen synthase kinase 3 mRNA, complete cds. | 300.802546 | 78.3361719 | 26% | 24% |
| 34592_at | Human ribosomal protein S17 mRNA, complete cds. | 6148.256 | 1601.95246 | 26% | 29% |
| 39782_at | H. sapiens mRNA for C1D protein. | 444.403273 | 115.889308 | 26% | 22% |
| 37031_at | Human mRNA for KIAA0183 gene, partial cds. | 213.145909 | 55.7684469 | 26% | 27% |
| 32644_at | Homo sapiens mRNA for KIAA0169 protein, partial cds. | 289.728273 | 75.8079662 | 26% | 31% |
| 31907_at | Homo sapiens mRNA for ribosomal protein L14, complete cds. | 2861.42573 | 748.942864 | 26% | 26% |
| 36676_at | Cluster Incl AL031659:dJ343K2.2.1 (ribophorin∥(isoform 1))/ | 628.458 | 165.061493 | 26% | 31% |
| | cds = (284,2179)/gb = AL031659/gi = 4468296/ug = Hs. 75722/ | | | | |
| | len = 2488 | | | | |
| 39050_at | Homo sapiens poly(A) binding protein∥(PABP2) gene, complete | 328.824273 | 86.4321681 | 26% | 30% |
| | cds. | | | | |
| 39711_at | Human 80K-H protein (kinase C substrate) mRNA, complete cds. | 286.512455 | 75.4567186 | 26% | 27% |
| 891_at | Homo sapiens GLI-Krupple related protein (YY1) mRNA, complete | 194.662273 | 51.4392188 | 26% | 30% |
| | cds. | | | | |
| 41765_at | pec1.2-4.D10.r ecnorm Homo sapiens cDNA 5′, mRNA sequence. | 2895.27546 | 765.872869 | 26% | 27% |
| 40281_at | Human mRNA for KIAA0158 gene, complete cds. | 1055.20136 | 279.345211 | 26% | 26% |
| | wp10g06.x1 NCI_CGAP_Kid12 Homo sapiens cDNA clone | | | | |
| 35850_at | IMAGE: 2464474 3′ similar to WP: F29B9.4 CE09782;, mRNA | 154.010818 | 40.7986435 | 26% | 23% |
| | sequence. | | | | |
| 35753_at | Homo sapiens mRNA for PRP8 protein, complete cds. | 846.212273 | 224.661951 | 27% | 22% |
| 39079_at | Homo sapiens mRNA for human protein homologous to DROER | 949.239727 | 252.309065 | 27% | 27% |
| | protein, complete cds. | | | | |
| 33706_at | Homo sapiens mRNA for SART-1, complete cds. | 264.590273 | 70.4664242 | 27% | 29% |
| 32438_at | Homo sapiens ribosomal protein S20 (RPS20) mRNA, complete cds. | 5665.22391 | 1509.6601 | 27% | 31% |
| 36942_at | Human mRNA for KIAA0174 gene, complete cds. | 302.519727 | 80.6550647 | 27% | 22% |
| 1394_at | Homo sapiens GTP-binding protein (rhoA) mRNA, complete cds. | 982.336 | 262.060927 | 27% | 22% |
| 1161_at | Human 90 kD heat shock protein gene, complete cds. | 3068.64864 | 818.752924 | 27% | 29% |
| 33657_at | Homo sapiens ribosomal protein L34 (RPL34) mRNA, complete cds. | 3003.20536 | 801.901766 | 27% | 26% |
| 36208_at | Human mRNA for KIAA9001 gene, complete cds. | 334.312727 | 89.3038991 | 27% | 29% |
| | wl57f04.x1 NCI_CGAP_Brn25 Homo sapiens cDNA clone | | | | |
| 39184_at | IMAGE: 2429023 3′ similar to TR: Q15370 Q15370 RNA | 781.212909 | 209.195608 | 27% | 24% |
| | POLYMERASE II TRANSCRIPTION FACTOR Sill P18 SUBUNIT;, | | | | |
| | mRNA sequence. | | | | |
| 37717_at | Human M4 protein mRNA, complete cds. | 568.950182 | 152.464818 | 27% | 28% |
| 1711_at | Human clone 53BP1 p53-binding protein mRNA, partial cds. | 132.636909 | 35.5821629 | 27% | 26% |
| 33913_at | Human HLA-B-associated transcript 2 (BAT2) mRNA, complete cds. | 448.727182 | 120.591553 | 27% | 31% |
| 39866_at | Homo sapiens mRNA for KIAA1063 protein, partial cds. | 872.284909 | 234.481854 | 27% | 25% |
| 777_at | Human rab GDI mRNA, complete cds. | 520.441909 | 140.415433 | 27% | 30% |
| 31538_at | Human acidic ribosomal phosphoprotein P0 mRNA, complete cds. | 4749.375 | 1281.756 | 27% | 28% |
| 36189_at | Human nuclear factor NF45 mRNA, complete cds. | 789.594091 | 213.32811 | 27% | 31% |
| 38297_at | H. sapiens mRNA for DRES9 protein. | 329.372091 | 88.9941743 | 27% | 24% |
| 38413_at | Human mRNA for DAD-1, complete cds. | 520.306818 | 140.88454 | 27% | 25% |
| 39342_at | H. sapiens mRNA for yeast methionyl-tRNA synthetase homologue. | 231.998182 | 62.8517235 | 27% | 29% |
| 1499_at | Human farnesyltransferase alpha-subunit mRNA, complete cds. | 439.475546 | 119.074695 | 27% | 25% |
| 32518_at | Homo sapiens zinc finger protein (ZPR1) mRNA, complete cds. | 236.681546 | 64.5653196 | 27% | 30% |
| 32573_at | Cluster Incl AL021546: Human DNA sequence from BAC 15E1 on | 1153.58136 | 315.372036 | 27% | 28% |
| | chromosome 12. Contains Cytochrome C Oxidase Polypeptide Vla- | | | | |
| | liver precursor gene, 605 ribosomal protein L31 pseudogene, pre- | | | | |
| | mRNA splicing factor SRp30c gene, two putative genes, ESTs, STSs | | | | |
| | and putative CpG islands/cds = (52,717)/gb = AL021546/gi = 2826890/ | | | | |
| | ug = Hs. 77608/len = 1069 | | | | |
| 31584_at | Human mRNA for translationally controlled tumor protein. | 2063.03309 | 564.60194 | 27% | 28% |
| 33485_at | Human mRNA for ribosomal protein, complete cds. | 3501.35264 | 958.722802 | 27% | 27% |
| 37040_at | Human mRNA for KIAA0088 gene, partial cds. | 753.565182 | 207.00012 | 27% | 24% |
| 34336_at | Homo sapiens mRNA for Lysyl tRNA Synthetase, complete cds. | 1035.29391 | 284.412474 | 27% | 23% |
| 38093_at | Human clone 23722 mRNA sequence. | 195.475909 | 53.830052 | 28% | 27% |
| 33674_at | H. sapiens mRNA for ribosomal protein L29. | 4179.38646 | 1151.09042 | 28% | 31% |
| 40824_at | Homo sapiens mRNA for KIAA0745 protein, partial cds. | 300.751727 | 83.0648844 | 28% | 23% |
| 905_at | Human guanylate kinase (GUK1) mRNA, complete cds. | 743.993 | 206.133175 | 28% | 27% |
| 31568_at | Human ribosomal protein S10 mRNA, complete cds. | 5036.81646 | 1397.18081 | 28% | 29% |
| 36928_at | H. sapiens OZF mRNA. | 640.350636 | 177.964095 | 28% | 27% |
| 36035_at | Homo sapiens mRNA for glycosylphosphatidylinositol anchor | 423.229546 | 117.771247 | 28% | 28% |
| | attachment 1 (GPAA1), complete cds. | | | | |
| 35307_at | Homo sapiens mRNA for GDP dissociation inhibitor beta. | 1048.41582 | 291.769146 | 28% | 23% |
| 34231_at | Homo sapiens histone acetyltransferase (HBO1) mRNA, complete | 156.493364 | 43.5749228 | 28% | 25% |
| | cds. | | | | |
| 868_at | Human TATA-binding protein associated factor 30 kDa subunit | 616.615546 | 172.223284 | 28% | 27% |
| | (taflI30) mRNA, complete cds. | | | | |
| 32576_at | Homo sapiens translation initiation factor 3 47 kDa subunit mRNA, | 1034.68636 | 289.205622 | 28% | 28% |
| | complete cds. | | | | |
| 38040_at | Homo sapiens splicing factor mRNA, complete cds. | 66.4970909 | 18.6011354 | 28% | 30% |
| 31955_at | H. sapiens fau mRNA. | 2940.44855 | 822.804695 | 28% | 25% |
| 36587_at | H. sapiens mRNA for elongation factor 2. | 2335.08527 | 714.544965 | 31% | 25% |
| 33875_at | PN001_AH_H03.r yodnorm Homo sapiens cDNA 5′, mRNA | 565.580636 | 178.228414 | 32% | 25% |
| | sequence. | | | | |
| 1310_at | Human mRNA for proteasome subunit HssC7-l, complete cds. | 1616.867 | 522.128796 | 32% | 28% |
| 41241_at | Homo sapiens mRNA for Asparaginyl tRNA Synthetase, complete | 510.672455 | 174.096791 | 34% | 25% |
| | cds. | | | | |
| 36167_at | Homo sapiens mRNA for proton-ATPase-like protein, complete cds. | 713.034818 | 250.985814 | 35% | 32% |
| 36138_at | Human mRNA for calcium dependent protease (small subunit). | 1228.93318 | 451.389619 | 37% | 27% |

TABLE 3A
Normal Tissue Summary

| Organ | Morphology | number of samples |
|---|---|---|
| ADIPOSE TISSUE | NORMAL TISSUE, NOS | 11 |
| AMYGDALOID NUCLEUS | NORMAL TISSUE, NOS | 1 |
| BLADDER, NOS | NORMAL TISSUE, NOS | 1 |
| BLOOD, NOS | NORMAL TISSUE, NOS | 6 |
| BLOOD, NOS | | 1 |
| BONES, NOS | DEGENERATION, NOS | 3 |
| BREAST, NOS | NORMAL TISSUE, NOS | 40 |
| CEREBELLUM, NOS | NORMAL TISSUE, NOS | 1 |
| CERVIX, NOS | CHRONIC INFLAMMATION, NOS | 1 |
| CERVIX, NOS | MORPHOLOGY UNKNOWN | 1 |
| CERVIX, NOS | NORMAL TISSUE, NOS | 35 |
| COLON, NOS | DILATATION, NOS | 1 |
| COLON, NOS | NORMAL TISSUE, NOS | 42 |
| CORTEX OF FRONTAL LOBE | NORMAL TISSUE, NOS | 2 |
| CORTEX OF PARIETAL LOBE | NORMAL TISSUE, NOS | 1 |
| CORTEX OF TEMPORAL LOBE | NORMAL TISSUE, NOS | 1 |
| DUODENUM, NOS | NORMAL TISSUE, NOS | 5 |
| ENDOCERVIX | SQUAMOUS METAPLASIA | 1 |
| ENDOMETRIUM, NOS | NORMAL TISSUE, NOS | 8 |
| ESOPHAGUS, NOS | NORMAL TISSUE, NOS | 4 |
| FALLOPIAN TUBE, NOS | NORMAL TISSUE, NOS | 3 |
| FIBROUS TISSUE | NORMAL TISSUE, NOS | 1 |
| GALLBLADDER, NOS | CHRONIC INFLAMMATION, NOS | 1 |
| GALLBLADDER, NOS | NORMAL TISSUE, NOS | 1 |
| KIDNEY, NOS | NO PATHOLOGIC DIAGNOSIS | 3 |
| KIDNEY, NOS | NORMAL TISSUE, NOS | 13 |
| LARYNX, NOS | NORMAL TISSUE, NOS | 2 |
| LEFT ATRIUM, NOS | NORMAL TISSUE, NOS | 29 |
| LIVER, NOS | NORMAL TISSUE, NOS | 7 |
| LIVER, NOS | | 1 |
| LUNG, NOS | NORMAL TISSUE, NOS | 25 |
| LYMPH NODE, NOS | NORMAL TISSUE, NOS | 5 |
| MUSCLES, NOS | NORMAL TISSUE, NOS | 7 |
| MYOMETRIUM, NOS | NORMAL TISSUE, NOS | 39 |
| OMENTUM, NOS | NORMAL TISSUE, NOS | 2 |
| OVARY, NOS | ATROPHY, NOS | 1 |
| OVARY, NOS | NORMAL TISSUE, NOS | 18 |
| PANCREAS, NOS | NORMAL TISSUE, NOS | 5 |
| PARATHYROID GLAND, NOS | NORMAL TISSUE, NOS | 1 |
| PLACENTA, NOS | NORMAL TISSUE, NOS | 1 |
| PROSTATE, NOS | NORMAL TISSUE, NOS | 3 |
| RECTUM, NOS | NORMAL TISSUE, NOS | 22 |
| RIGHT ATRIUM, NOS | NORMAL TISSUE, NOS | 24 |
| RIGHT VENTRICLE, NOS | NORMAL TISSUE, NOS | 30 |
| SKIN, NOS | NORMAL TISSUE, NOS | 24 |
| SMALL INTESTINE, NOS | NORMAL TISSUE, NOS | 16 |
| SPLEEN, NOS | NORMAL TISSUE, NOS | 10 |
| STOMACH, NOS | CHRONIC INFLAMMATION, NOS | 1 |
| STOMACH, NOS | NORMAL TISSUE, NOS | 18 |
| SUBSTANTIA NIGRA | NORMAL TISSUE, NOS | 1 |
| TESTIS, NOS | NORMAL TISSUE, NOS | 1 |
| THYMUS, NOS | NORMAL TISSUE, NOS | 23 |
| THYROID GLAND, NOS | CHRONIC INFLAMMATION, NOS | 1 |
| THYROID GLAND, NOS | NORMAL TISSUE, NOS | 5 |
| TONGUE, NOS | NORMAL TISSUE, NOS | 1 |
| TONSIL, NOS | LYMPHOID HYPERPLASIA, NOS | 10 |
| URETER, NOS | NORMAL TISSUE, NOS | 1 |
| UTERUS, NOS | ENDOMETRIOSIS, NOS | 1 |
| UTERUS, NOS | NORMAL TISSUE, NOS | 14 |
| VEIN, NOS | NORMAL TISSUE, NOS | 5 |
| VULVA, NOS | NORMAL TISSUE, NOS | 2 |
| WHITE BLOOD CELL, NOS | NORMAL TISSUE, NOS | 16 |
| | | 560 |

TABLE 3B
Diseased Tissue Summary

| | | No. of |
|---|---|---|
| Organ | Morphology | Samples |
| ADRENAL GLAND, NOS | ADENOMA, NOS | 1 |
| ADRENAL GLAND, NOS | ADRENAL CORTICAL CARCINOMA | 1 |
| ADRENAL GLAND, NOS | PHEOCHROMOCYTOMA, NOS | 2 |
| AMPULLA OF VATER | ADENOCARCINOMA, NOS | 2 |
| ARTERY, NOS | ATHEROSCLEROSIS, NOS | 1 |
| BLADDER, NOS | SPINDLE CELL CARCINOMA | 1 |
| BLADDER, NOS | TRANSITIONAL CELL CARCINOMA, NOS | 2 |
| BLOOD, NOS | MORPHOLOGY NOT APPLICABLE | 10 |
| BONES, NOS | DEGENERATION, NOS | 9 |
| BONES, NOS | GIANT CELL TUMOR OF BONE, NOS | 1 |
| BRAIN, NOS | CHRONIC INFLAMMATION, NOS | 1 |
| BRAIN, NOS | MENINGIOMA, NOS | 1 |
| BREAST, NOS | CYSTOSARCOMA PHYLLODES, NOS | 2 |
| BREAST, NOS | FIBROADENOMA, NOS | 2 |
| BREAST, NOS | FIBROCYSTIC DISEASE, NOS | 3 |
| BREAST, NOS | HYPERTROPHY, NOS | 1 |
| BREAST, NOS | INFILTRATING DUCT AND LOBULAR CARCINOMA | 2 |
| BREAST, NOS | INFILTRATING DUCT CARCINOMA | 110 |
| BREAST, NOS | INFILTRATING LOBULAR CARCINOMA | 10 |
| BREAST, NOS | INTRADUCTAL CARCINOMA, NOS | 3 |
| BREAST, NOS | MEDULLARY CARCINOMA, NOS | 2 |
| BREAST, NOS | MUCINOUS ADENOCARCINOMA | 1 |
| BREAST, NOS | NORMAL TISSUE, NOS | 3 |
| BREAST, NOS | PAPILLARY ADENOCARCINOMA, NOS | 1 |
| BREAST, NOS | | 7 |
| CEREBELLUM, NOS | ALZHEIMER'S NEUROFIBRILLARY DEGENERATION | 1 |
| CERVIX, NOS | ADENOCARCINOMA, NOS | 2 |
| CERVIX, NOS | CARCINOMA, NOS | 1 |
| CERVIX, NOS | CHRONIC INFLAMMATION, NOS | 3 |
| CERVIX, NOS | NEOPLASM, METASTATIC | 1 |
| CERVIX, NOS | NORMAL TISSUE, NOS | 1 |
| CERVIX, NOS | SQUAMOUS CELL CARCINOMA, NOS | 3 |
| COLON, NOS | ACUTE AND CHRONIC INFLAMMATION, NOS | 4 |
| COLON, NOS | ADENOCARCINOMA, NOS | 24 |
| COLON, NOS | ADENOMA, NOS | 2 |
| COLON, NOS | CHRONIC INFLAMMATION, NOS | 2 |
| COLON, NOS | DIVERTICULITIS, NOS | 1 |
| COLON, NOS | MUCINOUS ADENOCARCINOMA | 5 |
| CORTEX OF FRONTAL LOBE | ALZHEIMER'S NEUROFIBRILLARY DEGENERATION | 3 |
| ENDOMETRIUM, NOS | ADENOCARCINOMA, NOS | 18 |
| ENDOMETRIUM, NOS | CHRONIC INFLAMMATION, NOS | 1 |
| ENDOMETRIUM, NOS | CLEAR CELL ADENOCARCINOMA, NOS | 2 |
| ENDOMETRIUM, NOS | HYPERPLASIA, NOS | 1 |
| ENDOMETRIUM, NOS | MORPHOLOGY UNKNOWN | 1 |
| ENDOMETRIUM, NOS | MULLERIAN MIXED TUMOR | 1 |
| ENDOMETRIUM, NOS | NEOPLASM, MALIGNANT | 1 |
| ENDOMETRIUM, NOS | PAPILLARY SEROUS ADENOCARCINOMA | 4 |
| ESOPHAGUS, NOS | SQUAMOUS CELL CARCINOMA, NOS | 1 |
| ESOPHAGUS, NOS | | 2 |
| GALLBLADDER, NOS | ACUTE AND CHRONIC INFLAMMATION, NOS | 4 |
| GALLBLADDER, NOS | CHRONIC INFLAMMATION, NOS | 16 |
| KIDNEY, NOS | ACUTE AND CHRONIC INFLAMMATION, NOS | 1 |
| KIDNEY, NOS | CHRONIC INFLAMMATION, NOS | 2 |
| KIDNEY, NOS | CLEAR CELL ADENOCARCINOMA, NOS | 11 |
| KIDNEY, NOS | CYST, NOS | 1 |
| KIDNEY, NOS | GLOMERULOSCLEROSIS, NOS | 5 |
| KIDNEY, NOS | MALIGNANT LYMPHOMA, NOS | 1 |
| KIDNEY, NOS | ONCOCYTOMA | 3 |
| KIDNEY, NOS | RENAL CELL CARCINOMA | 10 |
| KIDNEY, NOS | TRANSITIONAL CELL CARCINOMA, NOS | 1 |
| KIDNEY, NOS | WILMS' TUMOR | 1 |
| LACRIMAL GLAND, NOS | SQUAMOUS CELL CARCINOMA, NOS | 1 |
| LARYNX, NOS | SQUAMOUS CELL CARCINOMA IN SITU, NOS | 1 |
| LARYNX, NOS | SQUAMOUS CELL CARCINOMA, NOS | 1 |
| LEFT VENTRICLE, NOS | NORMAL TISSUE, NOS | 2 |
| LEFT VENTRICLE, NOS | | 3 |
| LIVER, NOS | ADENOCARCINOMA, NOS | 1 |
| LIVER, NOS | ANGIOMYOSARCOMA | 1 |
| LIVER, NOS | ATRESIA, NOS | 1 |
| LIVER, NOS | CHRONIC INFLAMMATION, NOS | 1 |
| LIVER, NOS | FIBROSIS, NOS | 10 |
| LIVER, NOS | FOCAL NODULAR HYPERPLASIA | 2 |
| LIVER, NOS | HEPATOBLASTOMA | 1 |
| LIVER, NOS | HEPATOCELLULAR CARCINOMA, NOS | 3 |
| LIVER, NOS | INFLAMMATION, NOS | 2 |
| LUNG, NOS | ADENOCARCINOMA, NOS | 9 |
| LUNG, NOS | ADENOSQUAMOUS CARCINOMA | 1 |
| LUNG, NOS | CHRONIC INFLAMMATION, NOS | 1 |
| LUNG, NOS | COLLAPSE, NOS | 1 |
| LUNG, NOS | DILATATION, NOS | 1 |
| LUNG, NOS | EMPHYSEMA, NOS | 7 |
| LUNG, NOS | FIBROSIS, NOS | 1 |
| LUNG, NOS | NEOPLASM, MALIGNANT | 1 |
| LUNG, NOS | NEOVASCULARIZATION | 2 |
| LUNG, NOS | NEUROENDOCRINE CARCINOMA | 1 |
| LUNG, NOS | NORMAL TISSUE, NOS | 1 |
| LUNG, NOS | SPINDLE CELL SARCOMA | 1 |
| LUNG, NOS | SQUAMOUS CELL CARCINOMA, NOS | 6 |
| LUNG, NOS | | 1 |
| LYMPH NODE, NOS | ADENOCARCINOMA, NOS | 5 |
| LYMPH NODE, NOS | ATYPIA SUSPICIOUS FOR MALIGNANCY | 1 |
| LYMPH NODE, NOS | GRANULOMATOUS INFLAMMATION, NOS | 1 |
| LYMPH NODE, NOS | HODGKIN'S DISEASE, NOS | 3 |
| LYMPH NODE, NOS | INFILTRATING DUCT CARCINOMA | 1 |
| LYMPH NODE, NOS | LYMPHOID HYPERPLASIA, NOS | 2 |
| LYMPH NODE, NOS | MALIGNANT LYMPHOMA, NOS | 6 |
| LYMPH NODE, NOS | SIGNET RING CELL CARCINOMA | 1 |
| LYMPH NODE, NOS | SQUAMOUS CELL CARCINOMA, NOS | 2 |
| MEDIASTINUM, NOS | CARCINOMA, ANAPLASTIC, NOS | 1 |
| MEDIASTINUM, NOS | NEUROBLASTOMA, NOS | 1 |
| MEDIASTINUM, NOS | SCHWANNOMA, NOS | 1 |
| MEDULLA OF KIDNEY | CHRONIC INFLAMMATION, NOS | 1 |
| MESENTERY, NOS | ADENOCARCINOMA, NOS | 1 |
| MUSCLES, NOS | ATROPHY, NOS | 2 |
| MYOMETRIUM, NOS | ADENOCARCINOMA, METASTATIC, NOS | 1 |
| MYOMETRIUM, NOS | ATROPHY, NOS | 1 |
| MYOMETRIUM, NOS | ENDOMETRIOSIS, NOS | 2 |
| MYOMETRIUM, NOS | LEIOMYOMA, NOS | 26 |
| NASOPHARYNX, NOS | SQUAMOUS CELL CARCINOMA, NOS | 1 |
| OMENTUM, NOS | ADENOCARCINOMA, NOS | 2 |
| OMENTUM, NOS | PAPILLARY SEROUS ADENOCARCINOMA | 9 |
| OMENTUM, NOS | SIGNET RING CELL CARCINOMA | 1 |
| OVARY, NOS | ABSCESS | 1 |
| OVARY, NOS | ADENOCARCINOMA, NOS | 7 |
| OVARY, NOS | CARCINOID TUMOR, NOS (EXCEPT OF APPENDIX, M-82401) | 1 |
| OVARY, NOS | CARCINOMA, NOS | 1 |
| OVARY, NOS | CLEAR CELL ADENOCARCINOMA, NOS | 1 |
| OVARY, NOS | DYSGERMINOMA | 1 |
| OVARY, NOS | ENDOMETRIOID CYSTADENOFIBROMA, BORDERLINE MALIGNANCY | 1 |
| OVARY, NOS | GRANULOSA CELL TUMOR, NOS | 1 |
| OVARY, NOS | MUCINOUS CYSTADENOCARCINOMA, NOS | 2 |
| OVARY, NOS | MULLERIAN MIXED TUMOR | 2 |
| OVARY, NOS | PAPILLARY SEROUS ADENOCARCINOMA | 8 |
| OVARY, NOS | PAPILLARY SEROUS TUMOR OF LOW MALIGNANT POTENTIAL | 1 |
| OVARY, NOS | POLYCYSTIC CHANGE, NOS | 1 |
| OVARY, NOS | SEROUS CYSTADENOCARCINOMA, NOS | 3 |
| OVARY, NOS | SEROUS CYSTADENOFIBROMA | 1 |
| OVARY, NOS | STRUMA OVARII, NOS | 2 |
| OVARY, NOS | THECOMA, NOS | 2 |
| PANCREAS, NOS | ADENOCARCINOMA, NOS | 10 |
| PANCREAS, NOS | CHRONIC INFLAMMATION, NOS | 2 |
| PANCREAS, NOS | MICROCYSTIC ADENOMA | 1 |
| PANCREAS, NOS | SCLEROSING INFLAMMATION, NOS | 1 |
| PARATHYROID GLAND, NOS | ADENOMA, NOS | 1 |
| PAROTID GLAND, NOS | CARCINOMA IN PLEOMORPHIC ADENOMA | 1 |
| PAROTID GLAND, NOS | WARTHIN'S TUMOR | 1 |
| PERITONEUM, NOS | PAPILLARY SEROUS ADENOCARCINOMA | 1 |
| PERITONEUM, NOS | SARCOMA, NOS | 1 |
| PROSTATE, NOS | ADENOCARCINOMA, NOS | 5 |
| PROSTATE, NOS | NODULAR HYPERPLASIA | 12 |
| RECTUM, NOS | ADENOCARCINOMA IN SITU, NOS | 1 |
| RECTUM, NOS | ADENOCARCINOMA, NOS | 20 |
| RECTUM, NOS | ADENOMA, NOS | 1 |
| RECTUM, NOS | CHRONIC INFLAMMATION, NOS | 3 |
| RECTUM, NOS | MUCINOUS ADENOCARCINOMA | 1 |
| SALIVARY GLAND, NOS | ADENOID CYSTIC CARCINOMA | 1 |
| SKIN, NOS | ADNEXAL TUMOR | 1 |
| SKIN, NOS | BASAL CELL CARCINOMA, NOS | 4 |
| SKIN, NOS | HEMANGIOMA, NOS | 1 |
| SKIN, NOS | MALIGNANT MELANOMA, NOS | 1 |
| SKIN, NOS | MELANOCYTIC HYPERPLASIA | 1 |
| SKIN, NOS | SQUAMOUS CELL CARCINOMA, NOS | 4 |
| SMALL INTESTINE, NOS | ADENOCARCINOMA, NOS | 1 |
| SMALL INTESTINE, NOS | MALIGNANT LYMPHOMA, NOS | 2 |
| SOFT TISSUES, NOS | ANGIOSARCOMA | 2 |
| SOFT TISSUES, NOS | CARCINOMA IN PLEOMORPHIC ADENOMA | 1 |
| SOFT TISSUES, NOS | FIBROMA, NOS | 1 |
| SOFT TISSUES, NOS | FIBROMATOSIS, NOS | 1 |
| SOFT TISSUES, NOS | FIBROUS HISTIOCYTOMA, MALIGNANT | 4 |
| SOFT TISSUES, NOS | HEMANGIOMA, NOS | 1 |
| SOFT TISSUES, NOS | LEIOMYOSARCOMA, NOS | 2 |
| SOFT TISSUES, NOS | LIPOMA, NOS | 3 |
| SOFT TISSUES, NOS | LIPOMATOSIS, NOS | 1 |
| SOFT TISSUES, NOS | LIPOSARCOMA, NOS | 2 |
| SOFT TISSUES, NOS | SQUAMOUS CELL CARCINOMA, NOS | 1 |
| SOFT TISSUES, NOS | SYNOVIAL SARCOMA, NOS | 1 |
| SPLEEN, NOS | ABERRANT TISSUE, NOS | 2 |
| SPLEEN, NOS | CHRONIC MYELOID LEUKEMIA | 3 |
| SPLEEN, NOS | GRANULOMATOUS INFLAMMATION, NOS | 1 |
| SPLEEN, NOS | HYPERTROPHY, NOS | 1 |
| SPLEEN, NOS | MALIGNANT LYMPHOMA, NOS | 2 |
| STOMACH, NOS | ADENOCARCINOID TUMOR | 1 |
| STOMACH, NOS | ADENOCARCINOMA, NOS | 21 |
| STOMACH, NOS | ATYPIA SUSPICIOUS FOR MALIGNANCY | 1 |
| STOMACH, NOS | CARCINOMA, NOS | 1 |
| STOMACH, NOS | CHRONIC INFLAMMATION, NOS | 8 |
| STOMACH, NOS | HYPERTROPHY, NOS | 1 |
| STOMACH, NOS | SIGNET RING CELL CARCINOMA | 2 |
| STOMACH, NOS | | 1 |
| SYNOVIUM OF JOINT, NOS | PROLIFERATION, NOS | 1 |
| TESTIS, NOS | MIXED GERM CELL TUMOR | 1 |
| TESTIS, NOS | SEMINOMA, NOS | 2 |
| THYMUS, NOS | ATROPHY, NOS | 1 |
| THYMUS, NOS | LYMPHOID HYPERPLASIA, NOS | 1 |
| THYROID GLAND, NOS | CARCINOMA, ANAPLASTIC, NOS | 1 |
| THYROID GLAND, NOS | CHRONIC INFLAMMATION, NOS | 5 |
| THYROID GLAND, NOS | FOLLICULAR ADENOCARCINOMA, NOS | 1 |
| THYROID GLAND, NOS | MALIGNANT LYMPHOMA, NOS | 1 |
| THYROID GLAND, NOS | NODULAR HYPERPLASIA | 16 |
| THYROID GLAND, NOS | PAPILLARY CARCINOMA, NOS | 4 |
| TONGUE, NOS | SQUAMOUS CELL CARCINOMA, NOS | 3 |
| TONSIL, NOS | LYMPHOID HYPERPLASIA, NOS | 10 |
| UTERUS, NOS | ADENOCARCINOMA, NOS | 1 |
| VULVA, NOS | SQUAMOUS CELL CARCINOMA, NOS | 5 |
| WHITE BLOOD CELL, NOS | | 7 |
| | | 695 |

TABLE 4

| AFFX | | | | |
|---|---|---|---|---|
| fragment | | Forward Primer | Reverse Primer | TaqMan probe |
| ID | Gene Name | (Name/sequence) | (Name/sequence | (Name/sequence |
| 39360_at | sorting nexin 3 | AF034546-83F/ | af034546-201R/ | af034546-112T/ |
| | | AAGCCGCAGAAC | ACCCTGATTTC | ACCCCCCAGCAA |
| | | CTGAATGA | GTAAGTGGTGA | CTTCCTCGAGAT |
| | | | A | C |
| 36027_at | polymerase | AA418779- | aa418779- | aa418779-382T |
| | (RNA) II (DNA | 362Forward/ | 33Reverse/ | Sequence/ATCCCC |
| | directed) | AGGAACTCAAGG | CCCAGTCTTCA | ATCATCATTCGC |
| | polypeptide F | CCCGAAA | TAGCTCCCATC | CGTTACC |
| | | | T | |
| 34849_at | seryl-tRNA | x91257- | x91257- | x91257-1278T/ |
| | synthetase | 1254Forward/ | 1342Reverse/ | CCAGGCTCGCCG |
| | | CTCCTGTTCTAA | CAAACTCCACC | GCTTCGA |
| | | TTGCACGGATT | TTGTCCATCAT | |
| | | | C | |

1. A method of identifying at least one gene that is consistently expressed across different cell or tissue types in an organism, comprising:
(a) preparing gene expression profiles for different cell or tissue types from the organism;
(b) calculating a coefficient of variation for at least one gene in each of the profiles across the different cell or tissue types; and
(c) selecting any gene whose coefficient of variation indicates that the gene is consistently expressed across the different cell or tissue types.

2. A method of claim 1, wherein step (c) comprises identifying at least one gene with a coefficient of variation of less than about 40%.

3. A method of claim 1, wherein the different cell or tissue types comprise greater than about 10 different cell or tissue types.

4. A method of claim 1, wherein the different cell or tissue types comprise greater than about 25 different cell or tissue types.

5. A method of claim 1, wherein the different cell or tissue types comprise greater than about 50 different cell or tissue types.

6. A method of claim 3, wherein the cell or tissue types comprise normal and diseased cell or tissue types.

7. A method of claim 1, wherein the organism is a mammal or plant.

8. A method of claim 7, wherein the mammal is human, dog, rat, mouse or plant.

9. A method of claim 8, wherein the expression profiles are generated by querying a gene expression database for the expression level of at least one gene in different cell or tissue types from the organism or from a cell line.

10. A set of probes comprising at least two probes that specifically hybridize to a gene identified by the method of claim 1.

11. A set of probes according to claim 10, wherein the set comprises probes that specifically hybridize to at least about 10 genes.

12. A set of probes according to claim 10, wherein the set comprises probes that specifically hybridize to at least about 25 genes.

13. A set of probes according to claim 10, wherein the set comprises probes that specifically hybridize to at least about 50 genes.

14. A set of probes according to claim 10, wherein the set comprises probes that specifically hybridize to at least about 100 genes.

15. A set of probes according to claim 10, wherein the probes are attached to a single solid substrate.

16. A set of probes of claim 15, wherein the solid substrate is a chip.

17. A method of normalizing the data from a nucleic acid detection assay comprising:
(a) detecting the expression level for at least one gene in a nucleic acid sample; and
(b) normalizing the expression of said at least one gene with the detected expression of an control gene identified by the method of claim 1.

18. A method of claim 17, wherein step (b) comprises normalizing the expression level of said at least one gene with the expression levels of at least about 10 control genes.

19. A method of claim 17, wherein step (b) comprises normalizing the expression level of said at least one gene with the expression levels of at least about 25 control genes.

20. A method of claim 17, wherein step (b) comprises normalizing the expression level of said at least one gene with the expression levels of at least about 50 control genes.

21. A method of claim 17, wherein step (b) comprises normalizing the expression level of said at least one gene with the expression levels of at least about 100 control genes.

22. A method of claim 17, wherein the assay is quantitative.

23. A method of claim 17, wherein the assay is a hybridization reaction conducted on a solid substrate.

24. A method of claim 23, wherein the solid substrate is an oligonucleotide array.

25. A method of claim 24, wherein the array comprises oligonucleotide probes that are complementary to the control genes.

26. A method of claim 17, wherein the assay is a polymerase chain reaction.

27. A set of probes comprising at least two probes that specifically hybridize to a gene of Table 1 or Table 2.

28. A set of probes of claim 27, comprising probes that specifically hybridize to at least about 10 genes of Table 1 or Table 2.

29. A set of probes of claim 27, comprising probes that specifically hybridize to at least about 25 genes of Table 1 or Table 2.

30. A set of probes of claim 27, comprising probes that specifically hybridize to at least about 50 genes of Table 1 or Table 2.

31. A set of probes of claim 27, comprising probes that specifically hybridize to at least about 100 genes of Table 1 or Table 2.

32. A set of probes of claim 27, comprising probes that specifically hybridize to at least about 100 genes of Table 2.

33. A set of probes of claim 27, wherein the probes are attached to a single solid substrate.

34. A set of probes of claim 33, wherein the solid substrate is a chip.

35. A method of normalizing the data from a nucleic acid detection assay comprising:
(a) detecting the expression level for at least one gene in a nucleic acid sample; and
(b) normalizing the expression of said at least one gene with the detected expression of a control gene of Table 1 or Table 2.

36. A method of claim 35, wherein step (b) comprises normalizing the expression level of said at least one gene with the expression levels of at least about 10 control genes of Table 1 or Table 2.

37. A method of claim 35, wherein step (b) comprises normalizing the expression level of said at least one gene with the expression levels of at least about 25 control genes of Table 1 or Table 2.

38. A method of claim 35, wherein step (b) comprises normalizing the expression level of said at least one gene with the expression levels of at least about 50 control genes of Table 1 or Table 2.

39. A method of claim 35, wherein step (b) comprises normalizing the expression level of said at least one gene with the expression levels of at least about 100 control genes of Table 1 or Table 2.

40. A method of claim 35, wherein the assay is quantitative.

41. A method of claim 35, wherein the assay is a hybridization reaction conducted on a solid substrate.

42. A method of claim 41, wherein the solid substrate is an oligonucleotide array.

43. A method of claim 42, wherein the array comprises oligonucleotide probes that are complementary to the control genes.

44. A method of claim 35, wherein the assay is a polymerase chain reaction.

45. A method of claim 17, wherein the normalizing of step (b) comprises dividing the expression level for said at least one gene by the detected expression level of said control gene.

46. A method of identifying at least one gene that is consistently expressed across different cell or tissue types in an organism or cell line, comprising:
(a) querying a gene expression database for the expression level of at least one gene in different cell or tissue types from the organism or cell lines;
(b) calculating a coefficient of variation for said at least one gene across the different cell or tissue types or cell lines; and
(c) identifying at least one gene whose coefficient of variation indicates that the gene is consistently expressed across the different cell or tissue types or cell lines.

47. A method of claim 46, wherein step (c) comprises identifying at least one gene with a coefficient of variation of less than about 40%.

48. A method of claim 47, wherein the different cell or tissue types comprise greater than about 10 different cell or tissue types.

49. A method of claim 47, wherein the different cell or tissue types comprise greater than about 25 different cell or tissue types.

50. A method of claim 47, wherein the different cell or tissue types comprise greater than about 50 different cell or tissue types.

51. A method of claim 46, wherein the cell or tissue types comprise normal and diseased cell or tissue types.

52. A method of claim 47, wherein the organism is a mammal or plant.

53. A method of claim 52, wherein the mammal is human, rat, mouse or plant.

54. A method of claim 53, wherein the mammal is human.
